(19) United States
(12) Patent Application Publication
(10) Pub. No.: US 2008/0027052 A1
(43) Pub. Date: Jan. 31, 2008

(54) METHODS FOR TREATING CYSTIC KIDNEY DISEASE

(76) Inventors: Sharon Moe, Indianapolis, IN (US); Vincent Gattone, Brownsburg, IN (US)

Correspondence Address: SOMMER BARNARD ATTORNEYS,P.C., ONE INDIANA SQ, SUITE 3500, INDIANAPOLIS, IN, 46204, US

(21) Appl. No.: 11/771,658

(22) Filed: Jun. 29, 2007

Related U.S. Application Data
(60) Provisional application No. 60/819,765, filed on Jul. 10, 2006.
(60) Provisional application No. 60/941,611, filed on Jun. 1, 2007.

(51) Int. Cl. A61K 31/136; A61K 31/135; A61K 31/196; A61K 31/4168; A61K 31/5377; A61P 13/12

(52) U.S. Cl. 514/233.8; 514/236.8; 514/401; 514/563; 514/646; 514/655; 514/789

(57) ABSTRACT

The present invention relates to methods for treating or preventing cystic kidney diseases or disorders using calcimimetics and pharmaceutically acceptable salts of these compounds. Various aspects related to treating mammal including humans. Still other aspects related to various formulations including combination formulation that may be used to treat kidney disease and to treat or prevent other pathologies such as pain, hypertension, water retention, infection, and the like.

### PRIORITY CLAIM

This application claims the benefit of U.S. Provisional Patent Application No., 60/819,765 filed on Jul. 10, 2006 and U.S. Provisional Patent Application No. 60/941,611 filed on Jun. 1, 2007, both of the Provisional Patent Applications are incorporated herein by reference in their entireties as if each were individually incorporated herein by reference in their entirety.

### FIELD OF THE INVENTION

This invention relates generally to the field of medicine and, more specifically, to methods for treating or preventing cystic kidney diseases or disorders.

### BACKGROUND OF THE INVENTION

Polycystic kidney disease (PKD) is the most common genetic kidney disorder, it affects between 1 in 400 and 1 in 1000 people worldwide and it is the third leading cause of kidney failure resulting in the need for kidney dialysis or transplantation (renal replacement therapies). Autosomal dominant polycystic kidney disease (ADKPD) is the most common form of PKD and is responsible for 85-90% of all PKD cases. The major manifestation of this disorder is the progressive cystic dilation of renal tubules Gabow P. et. al. (1990) Am J. Kidney Dis. 16:403-413. There are two types of this disease Type I, and Type II. Type I ADPKD is more severe than Type II; Type I is associated with earlier age of onset and renal disease. Symptoms of ADPKD typically develop between the ages of 30 and 40, but can begin in childhood. About 50% of patients with the disease eventually develop end-stage renal disease by about age 56, requiring kidney replacement therapies such as dialysis or kidney transplant. The number of patients requiring these therapies increases as patients age into their sixties and seventies. Autosomal recessive polycystic kidney disease (ARPKD) is a less common inherited form of the disease with an estimated incidence of 1 per 20,000 people. This form of PKD is an important cause of perinatal death because the kidneys enlarge and inhibit lung expansion, potentially to thee extent that the newborn's lungs cannot function. Most of the remaining children with this condition eventually develop renal failure and liver fibrosis often in the first decade of life. Acquired cystic kidney disease (ACKD) is a form of kidney disease that is not inherited, but develops in patients who have been on long-term dialysis treatment. Approximately 90% of people with ACKD have been on dialysis for five years or more.

There are also a number of other conditions associated with renal cysts which can progress to renal failure, for example, medullary cystic kidney disease (MCKD). This disease is an autosomal dominant hereditary condition typically appearing in adults and is relatively mild. Generally, in patients with MCKD cysts form only in the inner portion of the kidney (medulla) and both kidneys are shrunken in size. MCKD is characterized by salt wasting and polyuria. Familial nephronophthisis (NPH) is a recessive form of PKD which is more severe than MCKD, often leading to kidney failure in children. There are three basic types of NPH: infantile, juvenile, and adolescent. In patients wit NPH both kidneys are shrunken in size and renal cysts are usually found at the border of the medulla and cortex of the kidney. NPH is characterized by growth retardation, polyuria, salt, wasting, anemia, and progressive renal insufficiency.

All of the aforementioned renal cystic conditions are generally associated with the formation of cysts in various segments of the renal nephron. Cysts often begin as dilations or outpouchings from existing nephrons or collecting ducts or from the developmental counterparts of these structures. Renal cysts contain a fluid that presumably derives from their parent nephron and/or is a local secretion. In many of these renal cystic diseases pathology can also develop in several other organs including: liver, heart, vasculature and pancreas. However, in inherited renal cystic disease, the associated renal failure is usually a major contributor to the disease morbidity and mortality. In many of these conditions, it takes several years to a few decades to develop renal failure or terminal renal cystic disease.

In view of the severity and frequency of occurrence of cystic kidney diseases, there is a particular need for finding therapeutic agents useful in the prevention and treatment of these diseases.

### SUMMARY OF THE INVENTION

One aspect provides methods for using effective amounts of a calcimimetic compound or a pharmaceutically acceptable salt form of a calcimimetic compound to treat cystic kidney disease in a subject in need of such treatment. In some embodiments the subject is a mammal. In another embodiment, the subject is a human being. Various embodiments include treating forms of kidney disease including, but not limited to, autosomal dominate polycystic kidney disease (ADPKD), autosomal recessive polycystic kidney disease (ADPKD), acquired renal cystic disease (ARCD), medullary cystic kidney disease (MCKD), nephronophthisis, and the like.

In one embodiment the calcimimetic compound has the general Formula I, shown as follows, or a pharmaceutically acceptable salt form of the compound: 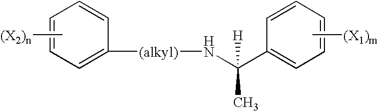
In which X₁ and X₂, may be identical or different, are each a radical chosen from CH₃, CH₃O, CH₃CH₂O, Br, Cl, F, CF₃, CHF₂, CH₂F, CF₃O, CH₃S, OH, CH₂OH, CONH₂, CN, NO₂, CH₃CH₂, propyl, isopropyl, butyl, isobutyl, t-butyl, acetoxy, and acetyl radicals, or two of X₁ may together form an entity chosen from, for example, fused cycloaliphatic rings, fused aromatic rings, and a methylene dioxy radical, or two of X₂ may together form an entity chosen from fused cycloaliphatic rings, fused aromatic rings, and a methylene dioxy radical; provided that X₂ is not a 3-t-butyl radical; n ranges from 0 to 5; m ranges from 1 to 5; and the alkyl radical is chosen from C₁-C₃ alkyl radicals, which are optionally substituted with at least one group chosen from saturated and unsaturated, linear, branched, and cyclic C₁-C₉ alkyl groups, dihydroindolyl and thiodihydroindolyl groups, and 2-, 3-, and 4-piperidinyl groups.

In one embodiment the Formula I calcimimetric compound can be, for example, N-(3-[2-chlorophenyl]-propyl)-R-α-methyl-3-methoxybenzylamine or its pharmaceutically acceptable salt form; alternatively, Formula I calcimimetric compound can be cinacalcet or a pharmaceutically acceptable salt form of the compound.

In another embodiment the calcimimetic compound used to treat kidney disease has general Formula II, shown as follows, or a pharmaceutically acceptable salt form of the compound: 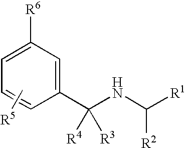
In which, R¹ is aryl, substituted aryl, heterocyclyl, substituted heterocyclyl, cycloalkyl, or substituted cycloalkyl; R² is alkyl or haloalkyl; R³ is H, alkyl, or haloalkyl; R⁴ is H, alkyl, or haloalkyl; each R⁵ present is independently selected form the group consisting of alkyl, substituted alkyl, alkoxy, substituted alkoxy, halogen, —C(═O)OH, —CN, —NR^{d}S(═O)ₘR^{d}, —NR^{d}C(═O)NR^{d}R^{d}, —NR^{d}S(═O)ₘNR^{d}R^{d}, or —NR^{d}C(═O)R^{d}; R⁶ is aryl, substituted aryl, heterocyclyl, substituted heterocyclyl, cycloalkyl, or substituted cycloalkyl; each R^{a} is, independently, H, alkyl, or haloalkyl; each R^{b} is, independently, aryl, aralkyl, heterocyclyl, or heterocyclylalkyl, each of which may be unsubstituted or substituted by up to 3 substituents selected from the group consisting of alkyl, halogen, haloalkyl, alkoxy, cyano, and nitro; each R^{c} is, independently, alkyl, haloalkyl, phenyl or benzyl, each of which may be substituted or unsubstituted, each R^{d} is, independently, H, alkyl, aryl, aralkyl, heterocyclyl, or heterocyclylalkyl wherein the alkyl, aryl, aralkyl, heterocyclyl, and heterocyclylalkyl are substituted by 0, 1, 2, 3 or 4 substituents selected from alkyl, halogen, alkoxy, cyano, nitro, R^{b}, —C(═O)R^{c}, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}S(═O)ₙR^{c} and —S(═O)ₙNR^{a}R^{a}; m is 1 or 2; n is 0, 1, or 2; and p is 0, 1, 2, 3, or 4; provided that if R² is methyl, p is 0, and R⁶ is unsubstituted phenyl, then R¹ is not 2,4-dihalophenyl, 2,4-dimethylphenyl, 2,4-diethylphenyl, 2,4,6-trihalophenyl, or 2,3,4-trihalophenyl. Formula II compound could be N-((6-(methoxyloxy)-4′-(trifluoromethyl)-1,1′-biphenyl-3-yl)methyl)-1-phenylethanamine, or its pharmaceutically acceptable salt form.

In still another embodiment the Formula II calcimimetic compound is, for example, (1R)-N-((6-chloro-3′-fluoro-3-biphenylyl)methyl)-1-(3-chlorophenyl)ethanamine, or its pharmaceutically acceptable salt form. In still another embodiment the calcimimetic compound according to Formula II is, for example, (1R)-1-(6-(methyloxy)-4′-(trifluoromethyl)-3-biphenylyl)-N-((1R)-1-phenylethyl)ethanamine, or its pharmaceutically acceptable salt form.

In yet another embodiment the calcimimetic compound has the general Formula III, shown as follows, or the compound is a pharmaceutically acceptable salt form of the compound: 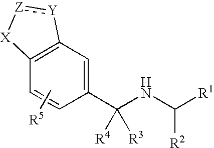
In which [symbol] represents a double or single bond; R¹ is R^{b}; R² is C₁₋₈alkyl or C₁₋₄haloalkyl; R³ is H, C₁₋₄haloalkyl or C₁₋₈alkyl; R⁴ is H, C₁₋₄haloalkyl or C₁₋₄alkyl; R⁵ is, independently, in each instance H, C₁₋₈alkyl, C₁₋₄haloalkyl, halogen, —OC₁₋₆alkyl, —NR^{a}R^{d} or NR^{d}C(═O)R^{d}; X is —CR^{d}═N—, —N═CR^{d}—, O, S or —NR^{d}—; when [symbol] is a double bond then Y is ═CR⁶— or ═N—and Z is —CR⁷═ or —N═; and when [symbol] is a single bond then Y is —CR^{a}R⁶— or —NR^{d}— and Z is —CR^{a}R⁷— or —NR^{d}—; and R⁶ is R^{d}, C₁₋₄haloalkyl, —C(═O)R^{c}, —OC₁₋₆alkyl, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, cyano, nitro, —NR^{a}S(═O)ₘR^{c} or —S(═O)ₘNR^{a}R^{a}; R⁷ is R^{d}, C¹⁻⁴haloalkyl, —C(═O)R^{c}, —OC₁₋₆alkyl, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, cyano, nitro, —NR^{a}S(═O)ₘR^{c} or —S(═O)ₘNR^{a}R^{a}; or R⁶ R⁷ together form a 3- to 6-atom saturated or unsaturated bridge containing 0, 1, 2 or 3 N atoms and 0, 1 or 2 atoms selected from S and O, wherein the bridge is substituted by 0, 1 or 2 substituents selected from R⁵; wherein when R⁶ and R⁷ form a benzo bridge, then benzo bridge may be additionally substituted by a 3- or 4-atoms bridge containing 1 or 2 atoms selected from N and O, wherein the bridge is substituted by 0 or 1 substituents selected from C₁₋₄alkyl; R^{a} is, independently, at each instance, H, C₁₋₄haloalkyl or C₁₋₆alkyl; R^{b} is, independently, at each instance, phenyl, benzyl, naphthyl or a saturated or unsaturated 5- or 6-membered ring heterocycle containing 1, 2 or 3 atoms selected from N, O and S, with no more than 2 of the atoms selected from O and S, wherein the phenyl, benzyl or heterocycle are substituted by 0, 1, 2 or 3 substituents selected from C₁₋₆alkyl, halogen, C₁₋₄haloalkyl, —OC₁₋₆alkyl, cyano and nitro; R^{c} is, independently, at each instance, C₁₋₆alkyl, halogen, C₁₋₄haloalkyl, phenyl or benzyl; R^{d} is, independently, at each instance, H, C₁₋₆alkyl, phenyl, benzyl or a saturated or unsaturated 5- or 6-membered ring heterocycle containing 1, 2 or 3 atoms selected from N, O and S, with no more than 2 of the atoms selected from O and S, wherein the C₁₋₆ alkyl, phenyl, benzyl, naphthyl and heterocycle are substituted by 0, 1, 2, 3 or 4 substituents selected from C₁₋₆alkyl, halogen, C₁₋₄haloalkyl, —OC₁₋₆alkyl, cyano and nitro, R^{b}, —OC(═O)R^{c}, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}C(═O)R^{c}, —NR^{a}S(═O)ₘR^{c} and —S(═O)ₘNR^{a}R^{a}; and m is 1 or 2,

In another embodiment the calcimimetic compound has the general Formula IV, shown as follows, or it is a pharmaceutically acceptable salt form of the compound: 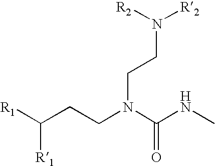
In which, R₁ and R′₁, may be the same or different, and represent an aryl radical, a heteroaryl radical, an aryl or heteroaryl radical substituted by one or more halogen atoms, by one or more hydroxy groups, by one or more linear or branched alkyl or alkoxy radicals containing from 1 to 5 carbon atoms, by one or more trifluoromethyl, trifluoromethoxy, —CN, —NO₂, acetyl, carboxyl, carboalkoxy or thioalkyl groups and the oxidised sulfoxide or sulfone forms thereof, thiofluoroalkoxy groups, or R₁ R′₁ form, with the carbon atom to which they are linked, a cycle of Formula: 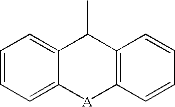
in which, A represents a single bond, a —CH₂— group, an oxygen, nitrogen or sulfur atom, R² and R′₂ form, with the nitrogen atom to which they are linked, a saturated heterocycle containing 4 or 5 carbons atoms optionally substituted by one or more linear or branched alkyl radicals containing from 1 to 5 carbon atoms, said heterocycle optionally containing a further heteroatom, itself being optionally substituted by a radical R₅ in which R₅ represents a hydrogen atom, a linear or branched alkyl radical containing from 1 to 5 carbon atoms, optionally substituted by an alkoxy or acyloxy radical, or R₂ and R′₂, which may be the same or different, represent a hydrogen atom, a linear or branched alkyl radical containing from 1 to 5 carbon atoms optionally substituted by a hydroxy or alkoxy radical containing from 1 to 5 carbon atoms, R₃ represents a thiazolyl, oxazolyl, benzothiazolyl or benzoxazolyl group of Formula: 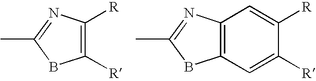
in which B represents an oxygen atom or a sulfur atom, in which R and R′, which may be the same or different, represent a hydrogen atom, a halogen atom, a hydroxy radical, a trifluoromethyl radical, a trifluoromethoxy radical, alkyl, alkoxy, alkoxycarbonyl or alkylthio radicals and the oxidised sulfoxide and sulfone form thereof linear or branched containing from 1 to 5 carbon atoms, an aryl or heteroaryl radical, an aryl or heteroaryl radical substituted by one or more groups selected from a halogen atom, a linear or branched alkyl radical containing from 1 to 5 carbon atoms, a trifluoromethyl radical, a trifluoromethoxy radical, a —CN group, an amino, dialkylamino and —NH—CO-alkyl group, an alkylthio group and the oxidised sulfoxide and sulfone form thereof, an alkylsulfonamide —NH—SO₂-alkyl group or by a morpholino group, or R and R′ on the thiazolyl or oxazolyl group can form a saturated or unsaturated cycle comprising or not comprising one or more optionally substituted heteroatoms, or it pharmaceutically acceptable salt form. Formula IV calcimimetic compound could be 3-(1,3-benzothiazol-2-yl)-1-(3,3-diphenylpropyl)-1-(2-(4-morpholinyl)ethyl)urea or its pharmaceutically acceptable salt form.

In still another embodiment the compound has the general Formula IV, as shown below, in one example, the calcimimetic compound according to Formula IV is N-(4-(2-((((3,3-diphenylpropyl)(2-(4-morpholinyl)ethyl)amino)carbonyl)amino)-1,3-thiazol-4-yl)phenyl)methanesulfonamide.

In still embodiment, the calcimimetic compound has the general Formula V, shown as follows, or it is a pharmaceutically acceptable salt form of the compound:

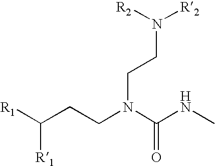

In which, R¹ is phenyl, benzyl, naphthyl or a saturated or unsaturated 5- or 6-membered heterocyclic ring containing 1, 2 or 3 atoms selected from N, O and S, with no more than 2 of the atoms selected from O and S, wherein the phenyl, benzyl, naphthyl or heterocyclic ring are substituted by 0, 1, 2 or 3 substituents selected from C₁₋₆alkyl, halogen, C₁₋₄haloalkyl, —C₁₋₆alkyl, cyano and nitro; R² is C₁₋₈alkyl or C₁₋₄haloalkyl; R³ is H, C₁₋₄haloalkyl or C₁₋₈alkyl; R⁴ is H, C₁₋₄haloalkyl or C₁₋₈alkyl; R⁵ is, independently, in each instance, H, C₁₋₈alkyl, C₁₋₄haloalkyl, halogen, —OC₁₋₆alkyl, —NR^{a}R^{d}, NR^{a}R^{d}, NR^{a}C(═O)R^{d}, substituted or unsubstituted pyrrolidinyl, substituted or unsubstituted azetidinyl, or substituted or unsubstituted piperidyl, wherein the substituents can be selected from halogen, —OR^{b}, —NR^{a}R^{d}, —C(═O)OR^{c}, —C(═O)NR^{a}R^{d}, —OC(═O)R^{c}, —NR^{a}C(═O)R^{c}, cyano, nitro, —NR^{a}S(═O)ₙR^{c} or —S(═O)ₙNR^{a}R^{d}; L is —O—, —OC₁₋₆alkyl-, —C₁₋₆alkylO—, —N(R^{a})(R^{d})—, —NR^{a}C(═O)—, —C(═O)—, —C(═O)NR^{d}C₁₋₆alkyl-, —C₁₋₆alkyl-C(═O)NR^{d}—, —NR^{d}C(═O)NR^{d}—, —NR^{d}C(═O)NR^{d}C₁₋₆alkyl-, —NR^{a}C(═O)R^{c}—, —NR^{a}C(═O)OR^{c}—, —OC₁₋₆alkyl-C(═O)O—, —NR^{d}C₁₋₆alkyl-, —C₁₋₆alkylNR^{d}—, —S—, —S(═O)ₙ—, —NR^{a}S(═O)ₙ, or —S(═O)ₙN(R^{a})—; Cy is a partially or fully saturated or unsaturated 5-8 membered monocyclic, 6-12 membered bicyclic, or 7-14 membered tricyclic ring system, the ring system formed of carbon atoms optionally including 1-3 heteroatoms if monocyclic, 1-6 heteroatoms if bicyclic, or 1-9 heteroatoms if tricyclic, and wherein each ring of the ring system is optionally substituted independently with one or more substituents of R⁶, C₁₋₈alkyl, C₁₋₄haloalkyl, halogen, cyano, nitro, —OC₁₋₆alkyl, —NR^{a}R^{d}, NR^{d}C(═O)R^{d}, —C(═O)OR^{c}, —C(═O)NR^{a}R^{d}, —OC(═O)R^{c}, —NR^{a}C(═O)R^{c}, —NR^{a}S(═O)ₘR^{d} or —S(═O)ₘNR^{a}R^{d}; R⁶ is a partially or fully saturated or unsaturated 5-8 membered monocyclic, 6-12 membered bicyclic, or 7-14 membered tricyclic ring system, the ring system formed of carbon atoms optionally including 1-3 heteroatoms if monocyclic, 1-6 heteroatoms if bicyclic, or 1-9 heteroatoms if tricyclic, and wherein each ring of the ring system is optionally substituted independently with one or more substituents of C₁₋₈alkyl, C₁₋₄haloalkyl, halogen, cyano, nitro, —OC₁₋₆alkyl, —NR^{a}R^{d}, NR^{d}(═O)R^{d}, —(═O)OR^{c}, —C(═O)NR^{a}R^{d}, —OC(═O)R^{c}, —NR^{a}C(═O)R^{c}, —NR^{a}S(═O)ₘR^{c} or —S(═O)ₘNR^{a}R^{d}; R^{a} is, independently, at each instance, H, C₁₋₄haloalkyl, C₁₋₆alkyl, C₁₋₄alkenyl, C₁₋₆alkylaryl or arylC₁₋₆alkyl; R^{b} is, independently, at each instance, C₁₋₈alkyl, C₁₋₄haloalkyl, phenyl, benzyl, naphthyl or a saturated or unsaturated 5- or 6-membered heterocyclic ring containing 1, 2 or 3 atoms selected from N, O and S, with no more than 2 of the atoms selected from O and S, wherein the phenyl, benzyl, naphthyl or heterocyclic ring are substituted by 0, 1, 2 or 3 substituents selected from C₁₋₆alkyl, halogen, C₁₋₄haloalkyl, —OC₁₋₆alkyl, cyano and nitro; R^{c} is, independently, at each instance, C₁₋₆alkyl, C₁₋₄haloalkyl, phenyl or benzyl; R^{d} is, independently, at each instance, H, C₁₋₆alkyl, C₁₋₆alkenyl, phenyl, benzyl, naphthyl or a saturated or unsaturated 5- or 6-membered heterocycle ring containing 1, 2 or 3 atoms selected from N, O and S, with no more than 2 of the atoms selected from O and S, wherein the C₁₋₆alkyl, phenyl, benzyl, naphthyl, and heterocycle are substituted by 0, 1, 2, 3 or 4 substituents selected from C₁₋₆alkyl, halogen, C₁₋₄haloalkyl, —OC₁₋₆alkyl, cyano and nitro, R^{b}, —C(═O)R^{c}, —OR^{b}, —NR^{a}R^{b}, —C(═O)OR^{c}, —C(═O)NR^{a}R^{b}, —OC(═O)R^{c}, —NR^{a}C(═O)R^{c}, —NR^{a}S(═O)ₘR^{c} and —S(═O)ₘNR^{a}R^{a}; m is 1 or 2; n is 1 or 2;

provided that if L is —O— or —OC₁₋₆alkyl-, then Cy is not phenyl.

In one aspect, the invention provides a method for treating a cystic kidney disease, comprising administering to a subject in need of such a treatment a therapeutically effective amount of a calcimimetic compound or its pharmaceutically acceptable salt form. In one embodiment the cystic kidney disease treated using this method is autosomal dominant polycystic kidney disease (ADPKD). In still other embodiments the cystic kidney diseases treated using this method include, but are not limited to, acquired cystic kidney disease (ACKD), medullary cystic kidney disease (MCKD), nephronnophthisis (NPH), multicystic dysplasia, congenital cystic disease, Meckel syndrome, oro-facial-digital syndrome, tuberous sclerosis, Von Hippel-Landau syndrome, cerebro-renal-digital syndrome, genitopatellar syndrome or Bardt-Biedl syndrome.

Still another aspect provides a method for treating the above mentioned cystic kidney diseases by administering to a target subject not only therapeutically effective amount of the calcimimetric compound or its pharmaceutically acceptable salt from, but also pain medication including, but not limited to, NSAID, tramadol, clonidine, a narcotic, or an opioid.

Another aspect provides a method for treating the above mentioned cystic kidney diseases by administering to a target subject not only therapeutically effective amount of the calcimimetric compound or its pharmaceutically acceptable salt form, but also medication to reduce, for example, blood pressure. In one embodiment the medication administered to the subject to reduce blood pressure can be, for example, an antihypertensive medication or a diuretic.

Yet another aspect provides a method for treating the above mentioned cystic kidney diseases by administering to a target subject not only a therapeutically effect amount of a calcimimetric compound or its pharmaceutically acceptable salt form, but also an antibiotic, or an EGFR tyrosine kinase inhibitor.

Still another aspect provides a method for treating the above mentioned cystic kidney diseases by administering to a target subject not only a therapeutically effective amount of a calcimimetric compound or it pharmaceutically acceptable salt form, but also a surgical treatment.

A further aspect provides a method for treating the above mentioned cystic kidney diseases by administrating to a target subject not only therapeutically effective amount of the calcimimetric compound or it pharmaceutically acceptable salt form, but also a life style change or diet modification.

### BRIEF DESCRIPTION OF THE DRAWINGS

FIG. 1. Schematically represents the study design.

For FIGS. 2-6, Black bars are control group, light gray is Compound A, dark gray is Compound A+Calcium, dashed line bar=Calcium only Cpd=Compound.

FIG. 2. Change in BUN with treatment (change from 20 to 38)

FIG. 3. Illustrates the reduction in the cyst volume

FIG. 4. Illustrates the reduction in the cyst volume as a percent of body weight.

FIG. 5. Illustrates the reduction in fibrosis in the treatment groups.

FIG. 6. Illustrates the reduction in total fibrosis (Fibrosis score X kidney weight).

FIG. 7. Illustrates results from histomorphometric analysis of the kidneys of rats in the upper left panel (control), right upper panel (Compound A), left lower panel (Compound A+Ca), right lower panel (Ca).

### DETAILED DESCRIPTION OF THE INVENTION

I. Definitions

As used herein, the term “subject” is intended to mean a human, or an animal, in need of a treatment. This subject can have, or be at risk of developing, a kidney disease or disorders including for example various forms of cystic kidney diseases.

“Treating” or “treatment” of a disease includes; (1) preventing the disease, i.e., causing the clinical symptoms of the disease no to develop in a subject that may be or has been exposed to the disease or conditions that may cause the disease, or predisposed to the disease but does not yet experience or display symptoms of the disease, (2) inhibiting the disease, i.e., arresting or reducing the development of the disease or any of its clinical symptoms, or (3) relieving the disease, i.e., causing regression of the disease or any of its clinical symptoms.

The phrase “therapeutically effective amount” is the amount of the compound of the invention that will reduce the severity and/or the frequency of a given disease. Reducing the severity and/or frequency of a given disease includes arresting or reversing the disease, as well as slowing down the progression of the disease.

As used herein, “calcium sensing receptor” or “CaSR” refers to the G-protein-coupled receptor which senses and/or responds to changes in extracellular calcium and/or magnesium levels. Typically, activation of the CaSR produces rapid, transient increases in cytosolic calcium concentration by mobilizing calcium from thapsigargin-sensitive intracellular stores and by increasing calcium influx though voltage-insensitive calcium channels in the cell membrane (Brown et al., Nature 366: 575-580, 1993; Yamaguchi et al., Adv Pharmacol 47: 209-253, 2000).

Administration “in combination with” one or more further therapeutic agents includes simultaneous or concurrent administration and consecutive administration in any order of various therapeutic compounds.

The phrase “cystic kidney diseases” or “cystic renal diseases” refer to a group of diseases or disorders characterized by the presence of cysts distributed throughout at least one kidney but is not limited to polycystic kidney diseases (PKD), including autosomal dominant polycystic kidney disease (ADKPD), autosomal recessive polycystic kidney disease (ARPKD), acquired cystic kidney disease (ACKD), medullary cystic kidney disease (MCKD), familial nephronophthisis (NPH) and the like.

II. Calcimimetics compounds and pharmaceutical compositions comprising them, administration and dosage

A. Calcimimetic Compounds, Definitions

As used herein, the term “calcimimetic compound” or “calcimimetic” refers to a compound that binds to calcium sensing receptors and induces a conformational change that reduces the threshold for calcium sensing receptor activation by the endogenous ligand Ca²⁺. These calcimimetic compounds can also be considered allosteric modulators of the calcium receptors.

In one aspect, a calcimimetic may have one or more of the following activities: it evokes a transient increase in internal calcium, having a duration of less than 30 seconds (for example, by mobilizing internal calcium); it evokes a rapid increase in [Ca²⁺ᵢ], occurring within thirty seconds; it evokes a sustained increase (greater than thirty seconds) in [CA²⁺ᵢ] (for example, by causing an influx of external calcium); evokes an increase in inositol-1,4,5-triphosphate or diacylglycerol levels, usually within less than 60 seconds; and inhibits dopamine- or isoproterenol-stimulated cyclic AMP formation. In one aspect, the transient increase in [Ca²⁺ᵢ] can be abolished by pretreatment of the cell for ten minutes with 10 mM sodium fluoride or with an inhibitor of phospholipase C, or the transient increase is diminished by brief pretreatment (not more than ten minutes) of the cell with an activator of protein kinase C, for example, phorbol myristate acetate (PMA), mezerein or (−) indolactam V. In one aspect, a calcimimetic compound can be a small molecule. In another aspect, a calcimimetic can be an agonistic antibody to the CaSR.

Calcimimetic compounds useful in the present invention include those disclosed in, for example, European Patent No. 637,237, 657,029, 724,561, 787,122, 907,631, 933,354, 1,203,761, 1,235,797 1,258,471, 1,275,635, 1,281,702, 1,284,963, 1,296,142, 1,308,436, 1,509,497, 1,509,518, 1,553,078; International Publication Nos. WO 93/04373, WO 94/18959, WO 95/11221, WO 96/12697, WO 97/41090, WO 01/34562, WO 01/90069, WO 02/14259, WO 02/059102, WO 03/099776, WO 03/099814, WO 04/017908; WO 04/094362, WO 04/106280, WO06/117211; WO 06/123725; U.S. Pat. Nos. 5,688,938, 5,763,569, 5,962,314, 5,981,599, 6,001,884, 6,011,068, 6,031,003, 6,172,091, 6,211,244, 6,313,146, 6,342,532, 6,362,231, 6,432,656, 6,710,088, 6,750,255, 6,908,935, 7,157,498, 7,176,322 and U.S. Patent Application Publication No. 2002/0107406, 2003/0008876, 2003/0144526, 2003/0176485, 2003/0199497, 2004/0006130, 2004/0077619, 2005/0032796, 2005/0107448, 2005/0143426, European patent application PCT/EP2006/004166, and French patent application 0511940.

In certain embodiments, the calcimimetic compound is chosen from compounds of Formula I and pharmaceutically acceptable salts thereof:

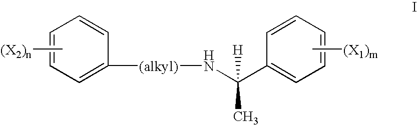

wherein

X₁ and X₂, which may be identical or different, are each a radical chosen from CH₃, CH₃O, CH₃CH₂O, Br, Cl, F, CF₃, CHF₂, CH₂F, CF₃O, CH₃S, OH, CH₂OH, CONH₂, CN, NO₂, CH₃CH₂, propyl, isopropyl, butyl, isobutyl, t-butyl, acetoxy, and acetyl radicals, or two of X₁ may together form an entity chosen from fused cycloaliphatic rings, fused aromatic rings, and a methylene dioxy radical, or two of X₂ may together form an entity chosen from fused cycloaliphatic rings, fused aromatic rings, and a methylene dioxy radical; provided that X₂ is not a 3-t-butyl radical;

n ranges from 0 to 5;

m ranges from 1 to 5; and

the alkyl radical is chosen from C1-C3 alkyl radicals, which are optionally substituted with at least one group chosen from saturated and unsaturated, linear, branched, and cyclic C1-C9 alkyl groups, dihydroindolyl and thiodihydroindolyl groups, and 2-, 3-, and 4-piperid(in)yl groups.

The calcimimetic compound may also be chosen from compounds of Formula II: 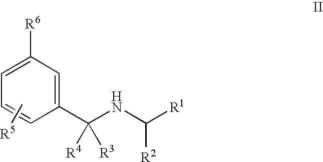
and pharmaceutically acceptable salts thereof,
wherein:

R¹ is aryl, substituted aryl, heterocyclyl, substituted heterocyclyl, cycloalkyl, or substituted cycloalkyl;

R² is alkyl or haloalkyl;

R³ is H, alkyl, or haloalkyl;

R⁴ is H, alkyl, or haloalkyl;

each R⁵ present is independently selected form the group consisting of alkyl, substituted alkyl, alkoxy, substituted alkoxy, halogen, —C(═O)OH, —CN, —NR^{d}S(═O)ₘR^{d}, —NR^{d}C(═O)NR^{d}R^{d}, —NR^{d}S(═O)ₘNR^{d}R^{d}, or —NR^{d}C(═O)R^{d};

R⁶ is aryl, substituted aryl, heterocyclyl, substituted heterocyclyl, cycloalkyl, or substituted cycloalkyl;

each R^{a} is, independently, H, alkyl, or haloalkyl;

each R^{b} is, independently, aryl, aralkyl, heterocyclyl, or heterocyclylalkyl, each of which may be unsubstituted or substituted by up to 3 substituents selected from the group consisting of alkyl, halogen, haloalkyl, alkoxy, cyano, and nitro;

each R^{c} is, independently, alkyl, haloalkyl, phenyl or benzyl, each of which may be substituted or unsubstituted;

each R^{d} is, independently, H, alkyl, aryl, aralkyl, heterocyclyl, or heterocyclylalkyl wherein the alkyl, aryl, aralkyl, heterocyclyl, and heterocyclylalkyl are substituted by 0, 1, 2, 3 or 4 substituents selected from alkyl, halogen, alkoxy, cyano, nitro, R^{b}, —C(═O)R^{c}, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}S(═O)ₙR^{c} and —S(═O)ₙNR^{a}R^{a};

m is 1 or 2;

n is 0, 1, or 2; and

p is 0, 1, 2, 3, or 4;

provided that if R² is methyl, p is 0, and R⁶ is unsubstituted phenyl, then R¹ is not 2,4-dihalophenyl, 2,4-dimethylphenyl, 2,4-diethylphenyl, 2,4,6-trihalophenyl, or 2,3,4-trihalophenyl. These compounds are described in detail in published US patent application No. 20050082625.

In one aspect, the calcimimetic compound can be N-((6-(methyloxy)-4′-(trifluoromethyl)-1,1′-biphenyl-3-yl)methyl)-1-phenylethanamine, or a pharmaceutically acceptable salt thereof. In another aspect, the calcimimetic compound can be XX AMG737, or a pharmaceutically acceptable salt thereof. In a further aspect, the calcimimetic compound can be XX AMG132, or a pharmaceutically acceptable salt thereof.

In certain embodiments of the invention the calcimimetic compound can be chosen from compounds of Formula III 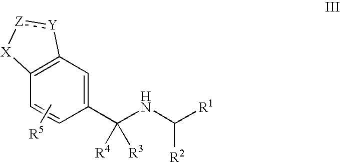
and pharmaceutically acceptable salts thereof, wherein:

[symbol] represents a double or single bond;

R¹ is R^{b};

R² is C₁₋₈alkyl or C₁₋₄haloalkyl;

R³ is H, C₁₋₄haloalkyl or C₁₋₈alkyl;

R⁴ is H, C₁₋₄haloalkyl or C₁₋₄alkyl;

R⁵ is, independently, in each instance H, C₁₋₈alkyl, C₁₋₄haloalkyl, halogen, —OC₁₋₆alkyl, —NR^{a}R^{d} or NR^{d}C(═O)R^{d};

X is —CR^{d}═N—, —N═CR^{d}—, O, S or —NR^{d}—;

when [symbol] is a double bond then Y is ═CR⁶— or ═N—and Z is —CR⁷═ or —N═; and when [symbol] is a single bond then Y is —CR^{a}R⁶— or —NR^{d}— and Z is —CR^{a}R⁷— or —NR^{d}—; and

R⁶ is R^{d}, C₁₋₄haloalkyl, —C(═O)R^{c}, —OC₁₋₆alkyl, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, cyano, nitro, —NR^{a}S(═O)ₘR^{c} or —S(═O)ₘNR^{a}R^{a};

R⁷ is R^{d}, C¹⁻⁴haloalkyl, —C(═O)R^{c}, —OC₁₋₆alkyl, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, cyano, nitro, —NR^{a}S(═O)ₘR^{c} or —S(═O)ₘNR^{a}R^{a}; or R⁶ and R⁷ together form a 3- to 6-atom saturated or unsaturated bridge containing 0, 1, 2 or 3 N atoms and 0, 1 or 2 atoms selected from S and O, wherein the bridge is substituted by 0, 1 or 2 substituents selected from R⁵; wherein when R⁶ and R⁷ form a benzo bridge, then the benzo bridge may be additionally substituted by a 3- or 4-atoms bridge containing 1 or 2 atoms selected from N and O, wherein the bridge is substituted by 0 or 1 substituents selected from C₁₋₄alkyl;

R^{a} is, independently, at each instance, H, C₁₋₄haloalkyl or C₁₋₆alkyl;

R^{b} is, independently, at each instance, phenyl, benzyl, naphthyl or a saturated or unsaturated 5- or 6-membered ring heterocycle containing 1, 2 or 3 atoms selected from N, O and S, with no more than 2 of the atoms selected from O and S, wherein the phenyl, benzyl or heterocycle are substituted by 0, 1, 2 or 3 substituents selected from C₁₋₆alkyl, halogen, C₁₋₄haloalkyl, —OC₁₋₆alkyl, cyano and nitro;

R^{c} is, independently, at each instance, C₁₋₆alkyl, C₁₋₄haloalkyl, phenyl or benzyl;

R^{d} is, independently, at each instance, H, C₁₋₆alkyl, phenyl, benzyl or a saturated or unsaturated 5- or 6-membered ring heterocycle containing 1, 2 or 3 atoms selected from N, O and S, with no more than 2 of the atoms selected from O and S, wherein the C₁₋₆ alkyl, phenyl, benzyl, naphthyl and heterocycle are substituted by 0, 1, 2, 3 or 4 substituents selected from C₁₋₆alkyl, halogen, C₁₋₄haloalkyl, —OC₁₋₆alkyl, cyano and nitro, R^{b}, —OC(═O)R^{c}, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}C(═O)R^{c}, —NR^{a}S(═O)ₘR^{c} and —S(═O)ₘNR^{a}R^{a}; and

m is 1 or 2.

Compounds of Formula III are described in detail in the patent application US20040077619.

In one aspect, a calcimimetic compound is N-(3-[2-chlorophenyl]-propyl)-R-α-methyl-3-methoxybenzylamine HCl (Compound A). In another aspect, a calcimimetic compound is N-((6-(methyloxy)-4′-(trifluoromethyl)-1,1′-biphenyl-3-yl(methyl)-1-phenylethanamine (Compound B).

In one aspect the calcimimetic compound of the invention can be chose from compounds of Formula IV 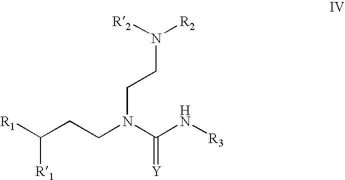
wherein:

Y is oxygen or sulphur;

R₁ and R′₁ are the same or different, and each represents an aryl group, a heteroaryl group, or R₁ and R′₁, together with the carbon atom to which they are linked, form a fused ring structure of Formula:

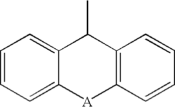

in which A represents a single bond, a methylene group, a dimethylene group, oxygen,

nitrogen or sulphur, said sulphur optionally being in the sulphoxide or sulphone forms, wherein each R₁ and R′₁, or said fused ring structure formed thereby, is optionally substituted by at least one substituent selected from the group c;

wherein the group c consists of: halogen atoms, hydroxyl, carboxyl, linear and branched alkyl, hydroxyalkyl, haloalkyl, alkylthio, alkenyl, and alkynyl groups; linear and branched alkoxyl groups; linear and branched thioalkyl groups; hydroxycarbonylalkyl; alkylcarbonyl; alkoxycarbonylalkyl; alkoxycarbonyl; trifluoromethyl; trifluoromethoxyl; —CN; —NO₂; alkylsulphonyl groups optionally in the sulphoxide or sulphone forms; wherein any alkyl component has from 1 to 6 carbon atoms, and any alkenyl or alkynyl components have from 2 to 6 carbon atoms;

and wherein, when there is more than one substituent, then each said substituent is the same or different;

R₂ and R′₂, which may be the same or different, each represents: a hydrogen atom; a linear or branched alkyl group containing from 1 to 6 carbon atoms and optionally substituted by at least one halogen atom, hydroxy or alkoxy group containing from 1 to 6 carbon atoms; an alkylaminoalkyl or dialkylaminoalkyl group wherein each alkyl group contains from 1 to 6 carbon atoms;

or R² and R′₂, together with the nitrogen atom to which they are linked, form a saturated or unsaturated heterocycle containing 0, 1 or 2 additional heteroatoms and having 5, 6, or 7 ring atoms, said heterocycle being optionally substituted by at least one substituent selected from the group ‘c’ defined above;

and wherein, when there is more than one substituent, said substituent is the same or different.

R₃ represents a group of Formula: 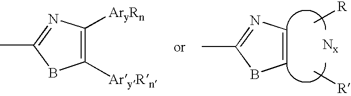
in which B represents an oxygen atom or a sulphur atom, x is 0, 1 or 2, y and y′ are the same or different, and each is 0 is 1, Ar and Ar′ are the same or different and each represents an aryl or heteroaryl group, n and n′ are the same or different, and each is 1, when the y or y′ with which it is associated is 0, or is equal to the number of positions that can be substituted on the associated Ar or Ar′ when the said y or y′ is 1, the fused ring containing N_{X} is a five- or six-membered heteroaryl ring, and wherein R and R′, which may be the same or different, each represent a hydrogen atom or a substituent selected from the group a;

wherein the group a consists of: halogen atoms; hydroxyl; carboxyl; aldehyde groups; linear and branched alkyl, alkenyl, alkynyl, hydroxyalkyl, hydroxyalkenyl, hydroxyalkynyl, haloalkyl, haloalkenyl, and haloalkynyl groups; linear and branched alkoxyl groups; linear and branched thioalkyl groups; aralkoxy groups; aryloxy groups; alkoxycarbonyl; aralkoxycarbonyl; aryloxycarbonyl; hydroxycarbonylalkyl; alkoxycarbonylalkyl; aralkoxycarbonylalkyl; aryloxycarbonylalkyl; perfluoroalkyl; perfluoroalkoxy; —CN; acyl; amino, alkylamino, aralkylamino, arylamino, dialkylamino, diaralkylamino, diarylamino, acylamino, and diacylamino groups; alkoxycarbonylamino, aralkoxycarbonylamino, aryloxycarbonylamino, alkylcarbonylamino, aralkylcarbonylamino, and arylcarbonylamino groups; alkylaminocarbonyloxy, aralylaminocarbonyloxy, and arylaminocarbonyloxy groups; alkyl groups substituted with an amino, alkylamino, aralkylamino, arylamino, dialkylamino, diaralkylamino, diarylamino, acylamino, trifluoromethylcarbonyl-amino, fluoroalkylcarbonylamino, or diacylamino group; CONH₂; alkyl-, aralkyl-, and aryl-amido groups; alkylthio, arylthio and aralkythio and the oxidised sulphoxide and sulphone forms thereof; sulphonyl, alkylsulphonyl, haloalkylsulphonyl, arylsulphonyl and aralkylsulphonyl groups; sulphonamide, alkylsulphonamide, haloalkylsulphonamide, di(alkylsulphonyl)amino, aralkylsulphonamide, di(aralkylsulphonyl)amino, arylsulphonamide, and di(arylsulphonyl)amino; and saturated and unsaturated heterocyclyl groups, said heterocyclyl groups being mono- or bicyclic and being optionally substituted by one or more substituents, which may be the same or different, selected from the group b;

wherein the group b consists of: halogen atoms; hydroxyl; carboxyl; aldehyde groups; linear and branched alkyl, alkenyl, alkynyl, hydroxyalkyl, hydroxyalkenyl, hydroxyalkynyl, haloalkyl, haloalkenyl, and haloalkynyl groups; linear and branched alkoxyl groups; linear and branched thioalkyl groups; alkoxycarbonyl; hydroxycarbonylalkyl; alkoxycarbonylalkyl; perfluoroalkyl; perfluoroalkoxy; —CN; acyl; amino, alkylamino, dialkylamino, acylamino, and diacylamino groups; alkyl groups substituted with an amino, alkylamino, dialkylamino, acylamino, or diacylamino group; CONH₂; alkylamido groups; alkylthio and the oxidised sulphoxide and sulphone forms thereof; sulphonyl, alkylsulphonyl groups; and sulphonamide, alkylsulphonamide, and di(alkylsulphonyl)amino groups;

wherein, in groups a and b, any alkyl components contain from 1 to 6 carbon atoms, and any alkenyl or alkynyl components contain from 2 to 6 carbon atoms, and are optionally substituted by at least one halogen atom or hydroxy group, and wherein any aryl component is optionally a heteroaryl group.

In one aspect, the calcimimetic compound can be 3-(1,3-benzothiazol-2-yl)-1-(3,3-diphenylpropyl)-1-(2-(4-morpholinyl)ethyl)urea or pharmaceutically acceptable salt thereof. In another aspect, the calcimimetic compound can be N-(4-(2-((((3,3-diphenylpropyl)(2-(4-morpholinyl)ethyl)amino)carbonyl)amino)-1,3-thiazol-4-yl)phenyl)methanesulfonamide or pharmaceutically acceptable salt thereof.

In one aspect, the calcimimetic compound of the invention can be chose from compounds of Formula V 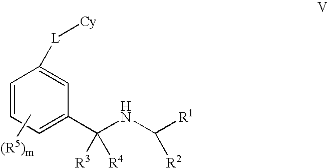
wherein:

R¹ is phenyl, benzyl, naphthyl or a saturated or unsaturated 5- or 6-membered heterocyclic ring containing 1, 2 or 3 atoms selected from N, O and S, with no more than 2 of the atoms selected from O and S, wherein the phenyl, benzyl, naphthyl or heterocyclic ring are substituted by 0, 1, 2 or 3 substituents selected from C₁₋₆alkyl, halogen, C₁₋₄haloalkyl, —OC₁₋₆alkyl, cyano and nitro;

R² is C₁₋₈alkyl or C₁₋₄haloalkyl;

R³ is H, C₁₋₄haloalkyl or C₁₋₈alkyl;

R⁴ is H, C₁₋₄haloalkyl or C₁₋₈alkyl;

R⁵ is, independently, in each instance, H, C₁₋₈alkyl, C₁₋₄haloalkyl, halogen, —OC₁₋₆alkyl, —NR^{a}R^{d}, NR^{a}R^{d}, NR^{a}C(═O)R^{d}, substituted or unsubstituted pyrrolidinyl, substituted or unsubstituted azetidinyl, or substituted or unsubstituted piperidyl, wherein the substituents can be selected from halogen, —OR^{b}, —NR^{a}R^{d}, —C(═O)OR^{c}, —C(═O)NR^{a}R^{d}, —OC(═O)R^{c}, —NR^{a}C(═O)R^{c}, cyano, nitro, —NR^{a}S(═O)ₙR^{c} or —S(═O)ₙNR^{a}R^{d};

L is —O—, —OC₁₋₆alkyl-, —C₁₋₆alkylO—, —N(R^{a})(R^{d})—, —NR^{a}C(═O)—, —C(═O)—, —C(═O)NR^{d}C₁₋₆alkyl-, —C₁₋₆alkyl-C(═O)NR^{d}—, —NR^{d}C(═O)NR^{d}—, —NR^{d}C(═O)NR^{d}C₁₋₆alkyl-, —NR^{a}C(═O)R^{c}—, —NR^{a}C(═O)OR^{c}—, —OC₁₋₆alkyl-C(═O)O—, —NR^{d}C₁₋₆alkyl-, —C₁₋₆alkylNR^{d}—, —S—, —S(═O)ₙ—, —NR^{a}S(═O)ₙ, or —S(═O)ₙN(R^{a})—;

Cy is a partially or fully saturated or unsaturated 5-8 membered monocyclic, 6-12 membered bicyclic, or 7-14 membered tricyclic ring system, the ring system formed of carbon atoms optionally including 1-3 heteroatoms if monocyclic, 1-6 heteroatoms if bicyclic, or 1-9 heteroatoms if tricyclic, and wherein each ring of the ring system is optionally substituted independently with one or more substituents of R⁶, C₁₋₈alkyl, C₁₋₄haloalkyl, halogen, cyano, nitro, —OC₁₋₆alkyl, —NR^{a}R^{d}, NR^{d}C(═O)R^{d}, —C(═O)OR^{c}, —C(═O)NR^{a}R^{d}, —OC(═O)R^{c}, —NR^{a}C(═O)R^{c}, —NR^{a}S(═O)ₘR^{c} or —S(═O)ₘNR^{a}R^{d};

R⁶ is a partially or fully saturated or unsaturated 5-8 membered monocyclic, 6-12 membered bicyclic, or 7-14 membered tricyclic ring system, the ring system formed of carbon atoms optionally including 1-3 heteroatoms if monocyclic, 1-6 heteroatoms if bicyclic, or 1-9 heteroatoms if tricyclic, and wherein each ring of the ring system is optionally substituted independently with one or more substituents of C₁₋₈alkyl, C₁₋₄haloalkyl, halogen, cyano, nitro, —OC₁₋₆alkyl, —NR^{a}R^{d}, NR^{d}(═O)R^{d}, —(═O)OR^{c}, —C(═O)NR^{a}R^{d}, —OC(═O)R^{c}, —NR^{a}C(═O)R^{c}, —NR^{a}S(═O)ₘR^{c} or —S(═O)ₘNR^{a}R^{d};

R^{a} is, independently, at each instance, H, C₁₋₄haloalkyl, C₁₋₆alkyl, C₁₋₄alkenyl, C₁₋₆alkylaryl or arylC₁₋₆alkyl;

R^{b} is, independently, at each instance, C₁₋₈alkyl, C₁₋₄haloalkyl, phenyl, benzyl, naphthyl or a saturated or unsaturated 5- or 6-membered heterocyclic ring containing 1, 2 or 3 atoms selected from N, O and S, with no more than 2 of the atoms selected from O and S, wherein the phenyl, benzyl, naphthyl or heterocyclic ring are substituted by 0, 1, 2 or 3 substituents selected from C₁₋₆alkyl, halogen, C₁₋₄haloalkyl, —OC₁₋₆alkyl, cyano and nitro;

R^{c} is, independently, at each instance, C₁₋₆alkyl, C₁₋₄haloalkyl, phenyl or benzyl;

R^{d} is, independently, at each instance, H, C₁₋₆alkyl, C₁₋₆alkenyl, phenyl, benzyl, naphthyl or a saturated or unsaturated 5- or 6-membered heterocycle ring containing 1, 2 or 3 atoms selected from N, O and S, with no more than 2 of the atoms selected from O and S, wherein the C₁₋₆alkyl, phenyl, benzyl, naphthyl, and heterocycle are substituted by 0, 1, 2, 3 or 4 substituents selected from C₁₋₆alkyl, halogen, C₁₋₄haloalkyl, —OC₁₋₆alkyl, cyano and nitro, R^{b}, —C(═O)R^{c}, —OR^{b}, —NR^{a}R^{b}, —C(═O)OR^{c}, —C(═O)NR^{a}R^{b}, —OC(═O)R^{c}, —NR^{a}C(═O)R^{c}, —NR^{a}S(═O)ₘR^{c} and —S(═O)ₘNR^{a}R^{a};

m is 1 or 2;

n is 1 or 2;

provided that if L is —O— or —OC₁₋₆alkyl-, then Cy is not phenyl.

In one aspect, the calcimimetic compound can be N-(2-chloro-5-((((1R)-1-phenylethyl)amino)methyl)phenyl)-5-methyl-3-isoxazolecarboxamide or a pharmaceutically acceptable salt thereof. In another aspect, the calcimimetic compound can be N-(2-chloro-5-((((1R)-1-phenylethyl)amino)methyl)phenyl)-2-pyridinecarboxamide or a pharmaceutically acceptable salt thereof.

Calcimimetric compounds useful in the methods of the invention include the calcimimetic compounds described above, as well as their steroisomers, enantiomers, polymorphs, hydrates, and pharmaceutically acceptable salts of any of the foregoing.

B. Methods of Assessing Calcimimetic Activity

In one aspect, compounds binding at the CaSR-activity modulating site can be identified using, for example, a labeled compound binding to the site in a competition-binding assay format.

Calcimimetic activity of a compound can be determined using techniques such as those described in International Publications WO 93/04373, WO 94/18959 and WO 95/11211.

Other methods that can be used to assess the calcimimetic activity of various compounds include those that are described below.

HEK 293 Cell Assay

HEK 293 cells engineered to express human parathyroid CaSR (HEK 293 4.0-7) have been described in detail previously (Nemeth E F et al. (1998) Proc. Natl. Acad. Sci. USA 95:4040-4045). This clonal cell line has been used extensively to screen for agonists, allosteric modulators, and antagonists of the CaSR (Nemeth E F et al. (2001) J. Pharmacol. Exp. Ther. 299:323-331).

For measurements of cytoplasmic calcium concentration, the cells are recovered from tissue culture flasks by brief treatment with 0.02% ethylenediaminetetraacetic acid (EDTA) in phosphate-buffered saline (PBS) and then washed and resuspended in Buffer A (126 mM NaCl, 4 mM KCl, 1 mM CaCl₂, 1 mM MgSO₄, 0.7 mM K₂HPO₄/KH₂PO₄, 20 mM Na-Hepes, pH 7.4) supplemented with 0.1% bovine serum albumin (BSA) and 1 mg/ml D-glucose. The cells are loaded with fura-2 by incubation for 30 minutes at 37° C. in Buffer A and 2 μM fura-2 acetoxymethylester. The cells are washed with Buffer B (Buffer B is Buffer A lacking sulfate and phosphate and containing 5 mM KCl, 1 mM MgCl₂, 0.5 mM CaCl₂ supplemented with 0.5% BSA and 1 mg/ml D-glucose) and resuspended to a density of 4 to 5×10⁶ cells/ml at room temperature. For recording fluorescent signals, the cells are diluted five-fold into prewarmed (37° C.) Buffer B with constant stirring. Excitation and emission wavelengths are 340 and 510 nm, respectively. The fluorescent signal is recorded in real time using a strip-chart recorder.

For fluorometric imaging plate reader (FLIPR) analysis, HEK 293 cells are maintained in Dulbecco's modified Eagle's medium (DMEM) with 10% fetal bovine serum (FBS) and 200 μg/ml hygromycin. At 24 hrs prior to analysis, the cells are trypsinized and plated in the above medium at 1.2×10⁵ cells/well in black sided, clear-bottom, collagen 1-coated, 96-well plates. The plates are centrifuged at 1,000 rpm for 2 minutes and incubated under 5% CO₂ at 37° C. overnight. Cells are then loaded with 6 μM fluo-3 acetoxymethylester for 60 minutes at room temperature. All assays are performed in a buffer containing 126 mM NaCl, 5 mM KCl, 1 mM MgCl₂, 20 mM Na-Hepes, supplemented with 1.0 mg/ml D-glucose and 1.0 mg/ml BSA fraction IV (pH 7.4).

In one aspect, the EC₅₀'s for the CaSR-active compounds can be determined in the presence of 1 mM Ca²⁺. The EC₅₀ for cytoplasmic calcium concentration can be determined starting at an extracellular Ca²⁺ level of 0.5 mM. FLIPR experiments can be performed using a laster setting of 0.8 W and a 0.4 second CCD camera shutter speed. Cells are challenged with calcium, CaSR-active compound or vehicle (20 μl) and fluorescence monitored at 1 second intervals for 50 seconds. Then a second challenge (50 μl) of calcium, CaSR-active compound, or vehicle can be made and the fluorescent signal monitored. Fluorescent signals are measured as the peak height of the response within the sample period. Each response is then normalized to the maximum peak observed in the plate to determine a percentage maximum fluorescence.

Bovine Parathyroid Cells

The effect of calcimimetic compounds on CaSR-dependent regulation of PTH secretion can be assessed using primary cultures of dissociated bovine parathyroid cells. Dissociated cells can be obtained by collagenase digestion, pooled, then resuspended in Percoll purification buffer and purified by centrifugation at 14,500×g for 20 minutes at 4° C. The dissociated parathyroid cells are removed and washed in a 1:1 mixture of Ham's F-12 and DMEM (F-12/DMEM) supplemented with 0.5% BSA, 100 U/ml penicillin, 100 μg/ml streptomycin, and 20 μg/ml gentamicin. The cells are finally resuspended in F-12/DMEM containing 10 U/ml penicillin, 10 μg/ml streptomycin, and 4 μg/ml gentamicin, and BSA was substituted with ITS+ (insulin, transferrin, selenous acid, BSA, and linoleic acid; Collaborative Research, Bedford, Mass.). Cells are incubated in T-75 flasks at 37° C. in a humidified atmosphere of 5% CO₂ in air.

Following overnight culture, the cells are removed from flasks by decanting and washed with parathyroid cell buffer (126 mM NaCl, 4 mM KCl, 1 mM MgSO₄, 0.7 mM K₂HPO₄/KH₂PO₄, 20 mM Na-Hepes, 20; pH 7.45 and variable amounts of CaCl₂ as specified) containing 0.1% BSA and 0.5 mM CaCl₂. The cells are resuspended in this same buffer and portions (0.3 ml) are added to polystyrene tubes containing appropriate controls, CaSR-active compound, and/or varying concentrations of CaCl₂. Each experimental condition is performed in triplicate. Incubations at 37° C. are for 20 minutes and can be terminated by placing the tubes on ice. Cells are pelleted by centrifugation (1500×g for 5 minutes at 4° C.) and 0.1 ml of supernatant is assayed immediately. A portion of the cells is left on ice during the incubation period and then processed in parallel with other samples. The amount of PTH in the supernatant from tubes maintained on ice is defined as “basal release” and subtracted from other samples. PTH is measured according to the vendor's instructions using rat PTH-(1-34) immunoradiometric assay kit (Immunotopics, San Clemente, Calif.).

MTC 6-23 Cell Calcitonin Release

Rat MTC 6-23 cells (clone 6), purchased from ATCC (Manassas, Va.) are maintained in growth media (DMEM high glucose with calcium/15% HIHS) that is replaced every 3 to 4 days. The cultures are passaged weekly at a 1:4 split ratio. Calcium concentration in the formulated growth media is calculated to be 3.2 mM. Cells are incubated in an atmosphere of 90% O₂/10% CO₂, at 37° C. Prior to the experiment, cells from sub-confluent cultures are aspirated and rinsed once with trypsin solution. The flasks are aspirated again and incubated at room temperature with fresh trypsin solution for 5-10 minutes to detach the cells. The detached cells are suspended at a density of 3.0×10⁵ cells/mL in growth media and seeded at a density of 1.5×10⁵ cells/well (0.5 mL cells suspension) in collagen-coated 48 well plates (Becton Dickinson Labware, Bedford, Mass.). The cells are allowed to adhere for 56 hours post-seeding, after which the growth media was aspirated and replaced with 0.5 mL of assay media (DMEM high glucose without/2% FBS). The cells are then incubated for 16 hours prior to determination of calcium-stimulated calcitonin release. The actual calcium concentration in this media is calculated to be less than 0.07 mM. To measure calcitonin release, 0.35 mL of test agent in assay media is added to each well and incubated for 4 hours prior to determination of calcitonin content in the media. Calcitonin levels are quantified according to the vendor's instructions using a rat calcitonin immunoradiometric assay kit (Immutopics, San Clemente, Calif.).

Inositol Phosphate Assay

The calcimimetic properties of compounds could also be evaluated in a biochemical assay performed on Chinese hamster ovarian (CHO) cells transfected with an expression vector containing cloned CaSR from rat brain [CHO(CaSR)] or not [CHO(WT)] Ruat M., Snowman A M., J. Biol. Chem 271, 1996, p 5972). CHO(CaSR) has been shown to stimulate tritiated inositol phosphate ([³H]IP) accumulation upon activation of the CaSR by Ca²⁺ and other divalent cations and by NPS 568 (Ruat et al., J. Biol. Chem 271, 1996). Thus, [³H]IP accumulation produced by 10 μM of each CaSR-active compound in the presence of 2 mM extracellular calcium can be measured and compared to the effect produced by 10 mM extracellular calcium, a concentration eliciting maximal CaSR activation (Dauban P. et al., Bioorganic & Medicinal Chemistry Letters, 10, 2000, p 2001).

A. Pharmaceutical Compositions and Administration

Calcimimetic compounds useful in the present invention can be used in the form of pharmaceutically acceptable salts derived from inorganic or organic acids. The salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecyclsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, mandelate, methansulfonate, nicotinate, 2-naphthalenesulfonate, oxalate, palmoate, pectinate, persulfate, 2-phenylpropionate, picrate, pivalate, propionate, salicylate, succinate, sulfate, tartrate, thiocyanate, tosylate, mesylate, and undecanoate. When compounds of the invention include an acidic function such as a carboxy group, then suitable pharmaceutically acceptable salts for the carboxy group are well known to those skilled in the art and include, for example, alkaline, alkaline earth, ammonium, quaternary ammonium cations and the like. For additional examples of “pharmacologically acceptable salts,” see Berge et al. J. Pharm. Sci. 66: 1, 1977. In certain embodiments of the invention salts of hydrochloride and salts of methanesulfonic acid can be used.

In some aspects of the present invention, the calcium-receptor active compound can be chosen form cinacalcet, i.e., N-(1-(R)-(1-naphthyl)ethyl]-3-[3-(trifluoromethyl)phenyl]-1-aminopropane, cinacalcet HCl, and cinacalcet methanesulfonate. The calcimimetic compound, such as cinacalcet HCl and cinacalcet methanesulfonate, can be in various forms such as amorphous powders, crystalline powders, and mixtures thereof. The crystalline powders can be in forms including polymorphs, pseudopolymorphs, crystal habits, micromeretics, and particle morphology.

For administration, the various compounds of the invention may be combines with adjuvants that are appropriate for the particular route of administration. For example, compounds according to various aspects of the invention may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, compounds may be tableted or encapsulated for conventional oral administration. Alternatively, the compounds useful in this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

Pharmaceutical compositions according to various aspects may be prepared in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, a granule and the like. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

The therapeutically effective amount of the calcium receptor-active compounds according to various aspects in various pharmaceutical formulations can range from about 0.1 mg to about 180 mg, for example from about 5 mg to about 180 mg, or from about 1 mg to about 100 mg of the calcimimetic compound per subject. In some aspects, the therapeutically effective amount of calcium receptor-active compound in the composition can be chosen from about 0.1 mg, about 1 mg, 5 mg, about 15 mg, about 20 mg, about 30 mg, about 50 mg, about 60 mg, about 75 mg, about 90 mg, about 120 mg, about 150 mg, about 180 mg.

While it may be possible to administer a calcium receptor-active compound to a subject alone, the compound administered will normally be present as an active ingredient in a pharmaceutical composition. Thus, a pharmaceutical composition of the invention may comprise a therapeutically effective amount of at least one calcimimetic compound, or an effective dosage amount of at least one calcimimetic compound.

As used herein, an “effective dosage amount” is an amount that provides a therapeutically effective amount of the calcium receptor-active compound when provided as a single dose, in multiple doses, or as a partial dose. Thus, an effective dosage amount of the calcium receptor-active compound of the invention includes an amount less than, equal to or greater than an effective amount of the compound; for example, a pharmaceutical composition in which two or more unit dosages, such as in tablets, capsules and the like, are required to administer an effective amount of the compound, or alternatively, a multidose pharmaceutical composition, such as powders, liquids and the like, in which an effective amount of the calcimimetic compound is administered by administering a portion of the composition.

Alternatively, a pharmaceutical composition in which two or more unit dosages, such as in tablets, capsules and the like, are required to administer an effective amount of the calcium receptor-active compound may be administered in less than an effective amount for one or more periods of time (e.g., a one-a-day administration, and a twice-a-day administration), for example to ascertain the effective dose for an individual subject, to desensitize an individual subject to potential side effects, to permit effective dosing readjustment or depletion of one or more other therapeutics administered to an individual subject, and/or the like.

The effective dosage amount of the pharmaceutical composition useful in the invention can range from about 1 mg to about 360 mg from a unit dosage form, for example about 5 mg, about 15 mg, about 30 mg, about 50 mg, about 60 mg, about 75 mg, about 90 mg, about 120 mg, about 150 mg, about 180 mg, about 210 mg, about 240 mg, about 300 mg, or about 360 mg from a unit dosage form.

In some aspects of the present invention, the compositions disclosed herein comprise a therapeutically effective amount of a calcium receptor-active compound for the treatment or prevention of bowel disorders. For example, in certain embodiments, the calcimimetic compound such as cinacalcet HCl can be present in an amount ranging form about 1% to about 70%, such as from about 5% to about 40%, from about 10% to about 30%, or from about 15% to about 20%, by weight relative to the total weight of the composition.

The compositions useful in the invention may contain one or more active ingredients in addition to the calcium sensing receptor-active compound. The additional active ingredient may be another calcimimetic compound, or it may be an active ingredient having a different therapeutic activity. Examples of such additional active ingredients include vitamins and their analogs, such as antibiotics, lanthanum carbonate, anti-inflammatory agents (steroidal and non-steroidal) and inhibitors of pro-inflammatory cytokine (ENBREL®, KINERET®). When administered as a combination, the therapeutic agents can be formulated as separate compositions that are given at the same time or different times, or the therapeutic agents can be given as a single composition.

In one aspect, the pharmaceutical compositions useful for methods of the invention may include additional compounds as described in more detail below.

In another aspect, the compounds used to practice the methods of the instant invention can be formulated for oral administration that release biologically active ingredients in the kidney. In one aspect, the compositions of the invention can be delivered to the kidneys using a prodrug, such as alkylylglucoside vector. Suzuki K. et al. (1999) Pharm. Res. 16: 1026-1034. In another aspect, the compositions of the invention can be coupled to substrates for renal specific enzymes, such as γ-glutamil derivatives. Drieman J. et al. (1990) J. Pharmacol. Exp. Ther. 252: 1255-1260. In a further aspect, the compounds and compositions of the invention can be delivered specifically to the kidneys using a low molecular weight protein (LMWP) approach. LMWP, such as lysozyme, are freely filtered proteins with a molecular weight of 30 kDa or less which accumulate specifically in the kidney, in particular in the proximal tubular cells through a luminal reabsorption mechanism. The physicochemical properties of the LMWP overrule that of the linked drug, further, the drug-LMWP conjugate is stable in the circulation, whereas after arrival in the kidney, the active drug is released in the catabolically active lysosomes of the proximal tubular cells. The compounds and compositions of the invention can be directly coupled to LMWPs via the lysine amino group of the protein forming an amide-bond. Alternatively, the drug can be coupled to the protein via different spacers such as oligopeptides (amide bond), (poly)-α-hydroxy acids (ester bond) and succinimidyloxycarbonyl-α-methyl-α-(2-pyridyldithio)toluene (disulfide bond). These drug-LMWP conjugates can be administered intravenously or subcutaneously. For more information, see Haas, M. et. al. (2002) Cadiovasc. Drugs and Ther. 16: 489-496. In other aspects, compositions of the invention can be delivered specifically to the kidney using a kidney surface application approach as described in Kawakami, S. et. al. (2002) Biol. Pharm. Bull. 25(7): 928-930.

III. Cystic Kidney Diseases

There are a number of kidney diseases and/or disorders characterized by the presence of cysts in at least one kidney. Some forms of these diseases are listed in Table 1 (see below).

TABLE 1

| | | | Transmission |
|---|---|---|---|
| Common name | Locus Name | Chromosome | Pattern |
| ADPKD | a. PKD1 | 16q13.3 | Autosomal |
| | | | Dominant (AD) |
| | b. PKD2 | 4q21-23 | AD |
| ARPKD | a. PKHD1 | 6p21.1-p12 | Autosomal |
| | | | Recessive (AR) |
| MCKD | a. MCKD1 | 1q21 | AD |
| | b. MCKD2 | 16p12 | AD |
| Nephronophthisis | a. NPHP1 | 2q13 | AR |
| | b. NPHP2 | 9q22 | AR |
| | c. NPHP3 | 3q21 | AR |
| | d. NPHP4 | 1p36 | AR |
| | e. NPHP5 | 3q13.33 | AR |
| Multicystic dysplasia | a. MCDK | ? | AR |
| Meckel Syndrome | a. MKS1 | 17q22-23 | AR |
| | b. MKS2 | 11q13 | AR |
| | c. MKS3 | 8q24 | AR |
| Oro-facio-digital syn | a. OFD1 | Xp22.2-22.3 | X-linked |
| | b. OFD2 | ? | AR |
| Tuberous sclerosis- | a. TSC1 | 9q34 | AD |
| | b. TSC2 | 16p13.3 | AD |
| von Hippel-Lindau | a. VHL | 3p | AD |
| Genitopatellar | a. ? | X | X-linked |
| syndrome | b. ? | ? | AR |
| Bardt-Biedl Syndrome | a. BBS1-6 | | AR |

The most common form of cystic kidney disease is Autosomal Dominant Polycystic Kidney Disease (ADPKD). Very early presentation of ADPKD can be very similar to typical ARPKD (Autosomal Recessive Polycystic Kidney Disease), and the radiological appearance of ARPKD kidneys at later stages can resemble that of ADPKD kidneys. Both disorders are characterized by increased proliferation and apoptosis of tubular epithelial cells. The development of renal cysts is generally associated with three factors: increased epithelial cell proliferation, epithelial cell secretion of fluid and altered extracellular matrix. While the cysts in ARPKD derive from collecting ducts, ADPKD cysts are thought to arise equally from all segments of the nephron and collecting ducts. Microdissections studies of ADPKD kidneys indicate that collecting ducts are diffusely enlarged and that collecting cysts are more numerous and larger than those derived from other tubular segments. Histological studies from ADPKD patients found that most cysts (particularly those 1 mm or more in diameter) stain positively for collecting duct markers. Furthermore, cultured epithelial cells from human ADPKD cysts exhibit a larger cyclic AMP (cAMP) response to desmopressin and vasopressin than to parathyroid hormone. This is consistent with these cells predominantly originating in the collecting duct. A feature common to ADPKD and ARPKD, and to animal models for these diseases, is a renal concentrating defect. A renal concentrating defect is characteristic of these types of diseases and may occur despite overexpression of the vasopressin V2 receptor and aquaporin 2 (AQP2) mRNA. Some human genes currently linked with renal cystic diseases are listed in Table 2 (see below).

TABLE 2

| Gene (protein) | Function |
|---|---|
| PKD1 (polycystin-1) | Receptor, ciliary sensor, cell adhesion |
| PKD2 (polycystin-2) | Calcium channel |
| PKHD1 (fybrocystin) | Transmembrane protein (receptor?) |
| NPHP1 (nephrocystin) | Cell-cell junction, ciliary function |
| INVS/NPHP2 (inversin) | Cell adhesion, Wnt signalling |
| NPHP3 (nephrocystin-3) | Ciliary function |
| NPHP4 (nephrocystin-4/ | Cell-cell junction, ciliary function |
| nephroretinin) | |
| NPHP5/IQCB1 (nephrocystin-5) | Ciliary function |
| BBS1-BBS8 | Basal body/centrosomal function |
| UMOD (uromodulin) | Bacterial adhesion/stone formation |
| | inhibitor* |
| OFD1 | Centosomal function |
| HNF-1β | Transcription factor |
| TSC1 (harmartin) | Rheb GTPase activating complex with |
| | tuberin |
| TSC2 (tuberin) | Rheb GTPase activating complex with |
| | hamartin |
| VHL (pVHL) | Targeting of HIF for degradation |

*Mechanism by which gene leads to cyst formation unknown.

For more information, see Torres, V. et. al. (2006) Nat. Clin. Practice vol. 2(1): 40-55. The invention provides methods for preventing and slowing down the progression of cystic kidney diseases. While individuals with early ADPKD often exhibit no symptoms, multiple cysts can often be detected in patients with this disease by age 20. The diagnosis of ADPKD can be strengthened by the presence of a family history of the disease or associated symptoms (infra) and can be definitive after genetic analysis of blood samples. Thus, if the objective is to prevent or slow down the progression of the renal cystic diseases, patients susceptible to the disease can be diagnosed by identifying mutations in the genes outlined in Table 2 or by early radiologic changes. While genetic testing can detect mutations before cysts even form, current versions of these tests cannot producibly predict the severity of cystic disease or age of onset of these diseases. Unborn babies of parents with PKD can be tested either with amniocentesis (evaluation of the amniotic fluid for genetic abnormalities) or by chorionic villus sampling (CVS), in which a small piece of the placenta is examined for genetic mutations. A genetic study of the DNA can confirm a diagnosis of PKD in individuals with the most common genetic variants.

The invention further provides therapeutic compositions and methods for the treatment of the kidney cystic diseases, wherein the beneficial or desired clinical results include, but are not limited to, alleviation of symptoms, diminishment of extent of disease, stabilized (i.e., not worsening) state of disease, delay or slowing down of disease progression, amelioration or palliation of the disease state, and remission, partial or total, whether detectable or undetectable. Some signs and symptoms of the cystic kidney diseases are described in more detail in Table 3.

TABLE 3

| Disease | Signs/Symptoms/History |
|---|---|
| ADPKD | Autosomal Dominant Polycystic Kidney Disease: Patients present in |
| | approximately the fourth decade of life with chronic flank pain, or |
| | imtermittent hematuria. Further symptoms include cyst hemorrage, renal |
| | infection, or nephrolithisasis. Hypertension and chronic renal failure are |
| | most commonly noted by the 5th decade of life and patients progress to |
| | end-stage renal disease (need for dialysis) in the 6th decade. However, the |
| | exact timing of the duration of disease and rate of progression to the need |
| | for dialysis varies. The number and severity of symptoms and |
| | complications tend to increase with age. These include hypertension, pain |
| | extrarenal cysts (e.g., hepatic or pancreatic cysts), intracranial aneurysms, |
| | cardiac valve disease, circulatory disturbances, and diverticular disease. |
| | The enlarged kidney and liver may be palpable. |
| ARPKD | Autosomal Recessive Polycystic Kidney Diseases: Symptoms develop |
| | before birth and may often lead to perinatal death. Surviving children |
| | usually develop kidney failure and hepatic fibrosis within a few years after |
| | birth. Severity varies from very severe, where babies may die within hours |
| | or days of birth, to less severe where there may be sufficient renal function |
| | for the child to survive a few to many years. At birth, children with |
| | ARPKD often exhibit severe bilaterally enlarged kidneys (large rigid |
| | masses can be felt on the back of both flanks), pulmonary hypoplasia |
| | (neonates can exhibit profound respiratory compromise). Infants may |
| | present with hypertension, diminshed urine concentrating abilty, and renal |
| | insufficiency. Growth retardation is reported in 25% of children. The |
| | disease may progress to renal failure and require dialysis or transplantation. |
| | With children aged 4-8 years (or older), the kidneys often are less severely |
| | affected, and a fibrotic hepatic pathology may predominate. Hepatic |
| | invlovement usually present with symptoms secondary to portal |
| | hypertension, such as varices and splenomegaly. Hepatic disease also may |
| | result in acute bacterial cholangitis or thrombocytopenia secondary to |
| | hypersplenism. Other complications that may occour include anemia, |
| | varicose veins and hemorrhoids |
| Nephronophthisis | Renal disease in children with infantile nephronopthisis may present as |
| (NPHP) - | failure to thrive, growth retardation, bone deformities, or hypocalcemic |
| medullary cysts. | tetany. Juvenile and adolescent forms may be present with polyuria |
| kidney disease | (secondary to concentrating diffilculties), polydipsia, anemia, and |
| complex | progressive renal failure. The autosomal recessive juvenile group also may |
| | have symptoms related to congenital hepatic fibrosis. Juveniles with renal- |
| | retinal syndrome may have ophthalmic disease. |
| Acquired renal | May be found in patients with all etiologies of end stage renal disease |
| cystic disease | (ESRD), particulary in patients who are dialysis dependent. The |
| (ARCD) | incidence, number, and size of cysts increase in proportion to the duration |
| | of dialysis. Most patients are asymptomatic, but symptoms may include |
| | gross hematuria, flank pain, renal colic, or a palpable renal mass. The |
| | presence of cysts confers an increased risk of develoning renal carcinoma. |
| | In addition, cysts may develop as a function of older age, but usually in |
| | limited number. |
| Tuberous | Clinical featues include facial nevi, cardiac rhabdomyomas, epilepsy, |
| sclerosis (TS) | angiofibromas, and mental retardation. Aproximately half of the patients |
| | have multiple renal angiomyolipomas. 20 to 25% of patients have some |
| | renal cysts, but diffuse renal cystic disease, which may result in chronic |
| | renal failure, is rare. |
| Von Hippel- | Clinical features include retinal and cerebellar hemangioblastomas, |
| Lindau syndrome | phechromocytomas, and cystic disease of the kidneys, pancreas, and |
| (VHLS) | epididymis. Renal carcinima may develop in as many as 40% of patients |

Cystic kidney diseases are generally diagnosed with an imaging-type study to identify the number, type and location of cysts. In addition, the kidney function is assessed by measuring blood urea nitrogen (BUN) and creatinine, and calculating or measuring clearance (i.e., creatinine clearance, eGFR (estimated GFR) formulas. In addition, a complete blood count is usually done, and elevations in the hematocrit may indicate elevated levels of erythropoietin secreted from cysts. Other measures of the magnitude of kidney dysfunction can be assessed by a serum chemistry profile including blood levels of bicarbonate, calcium and phosphorus, parathyroid hormone value test, urinalysis and/or urine culture.

The initial diagnosis of cystic kidney diseases is usually assessed by ultrasound, and the progression can be evaluated by overall kidney mass on MRI. Ultrasound of the kidneys or other organs where there may be cysts related to PKD is a very useful imaging technique and can detect cysts as small as 2 cm in size. Magnetic resonance imaging (MRI) and Cat Scan (CT scan) are more sensitive than ultrasound and can detect cysts as small as 1 cm in size. MRI is also helpful in identifying renal cell carcinoma that occurs more frequently among people with acquired cystic disease (ACKD). To reach a diagnosis of polycystic kidney disease, it is usually sufficient for the radiologist to visualize three or more cysts on the usually enlarged kidneys. When ADPKD is suspected, the physician will take a detailed family history of the patient to evaluate if anyone else in the family has a history of kidney cysts or other symptoms that may be compatible with the presence of ADPKD. For some of the other varieties of cystic kidney disease, especially nephronophthisis, a kidney biopsy may be needed to confirm a diagnosis.

IV. Methods of Treating Cystic Kidney Diseases Including PKD

In one aspect, the invention provides methods for the treatment of cystic kidney diseases. In one aspect, the invention provides methods for treatment of PKD. In one aspect, PKD includes autosomal dominant polycystic kidney disease (ADKPD), in another aspect, autosomal recessive polycystic kidney disease (ARPKD). In a further aspect, cystic kidney diseases include acquired cystic kidney disease (ACKD), medullary cystic kidney disease (MCKD) or familial nephronophthisis (NPHP). The invention provides materials and method for the treatment of various renal cystic disorders, including, but not limited to, those listed in Table 1 and Table 2.

Some aspects of the invention provide therapeutic compositions and methods for the treatment of cystic kidney diseases, wherein the beneficial or desired clinical results include, but are not limited to, alleviation of symptoms, diminishment of extent of disease, stabilized (i.e., not worsening) state of disease, delay or slowing down of disease progression, amelioration or palliation of the disease state, and remission, partial or total, whether readily detectable or currently undetectable. The development of renal cysts is generally associated with three factors: increased epithelial cell proliferation, epithelial cell secretion of fluid and altered extracellular matrix. Interventions have tried to focus on one or more of these factors in order to interrupt the cyst process. The proliferation and secretion appears to be associated with the misregulation of intracellular calcium and cyclic AMP in renal cystic epithelial cells. The lower calcium concentration in cystic epithelial cells appears to play a major role in the development of cAMP stimulated cell proliferation (and probably the secretion of cyst fluid as well).

Compounds and compositions of the present invention can be administered together with other medical treatments. In one aspect, compounds and compositions of the invention can be administered together with treatments to alleviate pain. In one aspect, these treatments can include benign therapies, such as hot compresses, massage, behavior modifications and physical therapy for spinal pain. In another aspect, the treatments can include drug therapy such as acetaminophen (e.g., Tylenol) and its narcotic combination formulations, tramadol, by itself or together with spinal blocks to prolong and intensify the effects of anesthesia, narcotics, administered orally or transdermally such as in Fentanyl patches, or injections of opioids into the area surrounding the spinal cord.

In one aspect, the methods of the invention can be practiced in conjunction with surgical treatments. In one aspect, the surgical treatment can include surgical drainage of cysts or cyst decompression to reduce the size of the cysts and the resulting pressure, e.g., decortication (unroofing and drainage), where cysts are opened and drained. In another aspect, the surgical treatment can be removal of kidney stones and/or-ureteral stones, such as extracorporeal shock wave lithotripsy, where sound waves are directed at the targeted stone reducing it to small particles that are easily passed through the urine. In a further aspect, the surgical treatments can be ureteroscopy, percutaneous surgery, or nephrectomy.

In one aspect, the methods of the invention can be practiced in combination with administration of medications to reduce blood pressure. In one aspect, the medications to reduce blood pressure can include antihypertensive medications, such as angiotensin converting enzyme (ACE) inhibitors (e.g., captopril, analapril, lisinopril) or angiotensin II receptor blockers (e.g., losartan, irbesartan, candesartan). In another aspect, the medications to reduce blood pressure can include diuretics (e.g., loop diuretics or thiazide diuretics). In another aspect, the methods of the invention can be practiced in combination with administration of medications or treat or prevent urinary tract infections, such as antibiotics. In one aspect, the methods of the invention can be practiced in combination with the treatment of anemia, (e.g. erythropoietin and derivatives), or blood transfusion. In one aspect, the methods of the invention can be practiced in combination with treatments associated with end stage renal replacement therapies, such as peritoneal dialysis or hemodialysis, continuous forms of dialysis or kidney transplant.

In a further aspect, the methods of the invention can be practiced in conjunction with dietary or lifestyle modifications, which may include a low-protein diet, reducing salt intake, drinking more water, caffeine avoidance, and cessation of smoking or heavy alcohol drinking.

In another aspect, the methods of the invention can be practiced in combination with administration of compounds and compositions that inhibit EGFR tyrosine kinase activity, and/or inhibit Her-2 (ErbB2 or neu) activity and/or with administration of compounds and compositions that inhibit ligand bioavailability. Examples of these compounds, such as EKB569 or WTACE 2, as described in Sweeney, W. et. al. (2003) Kidney Int. vol. 64, 1310-1319 and AG825 as described by Wilson S J et al., (2006) Biochim Biophys Acta, 1762:647-655. In a further aspect, the compounds and compositions of the invention can be administered together with vasopressin V2 receptor antagonists [Tolvaptan-(Otuska Pharmaceutical), lixivaptan (or VPA-985-CardioKine) or SR-121463 (Sanofi-Aventis)], in TOR inhibitors [Sirolimus (Rapamune, Rapamycin) or Everolimus (Novartis)], Somatostatin Agonists [(Octreotide LAR) or (Lanreotide, trades names Somatuline LA or Somatuline Autogel)], CDK inhibitors (roscovitine) or other statin therapeutics.

The following Examples are offered to more fully illustrate the invention, but are not to be construed as limiting the scope thereof.

### EXAMPLE 1

This Example outlines the methods used in the present invention to decrease cyst volume, kidney fibrosis, kidney size, and improve kidney function. These features are known to slow the progression of kidney deterioration in patients with kidney disease.

Animals

The Cy rat is a Han:SPRD rat with a spontaneous genetic mutation called Cy. These animals develop progressive kidney disease due to the formation of multiple kidney tubule cysts that arise from both the proximal and later the distal tubule. The colony of rats has been maintained through successive breeding of two heterozygous Cy/+ rats. This is an autosomal dominant gene, such that at birth, ¼ of the animals are normal, ½ are heterozygotes (Cy/+) and ¼ are homozygotes (Cy/Cy). Homozygotes (Cy/Cy) of either sex are easily identified after approximately 10 days of age by abdominal palpation and elevations in blood urea nitrogen (BUN), a finding used to verify parental heterozygosity for PKD. Homozygous Cy/Cy rats develop massively enlarged kidneys and severe azotemia and die by 4 weeks of age. Heterozygote male animals develop progressive chronic kidney disease (CKD) with rise in blood urea nitrogen (BUN) by 10 weeks and death from uremia by 40-50 weeks. Heterozygote female animals develop progressive CKD with rise in BUN not detected until 20 weeks, with death at 80 weeks.

Rats were housed in open top, shoebox cages and had free access to tap water and rat chow, either standard chow or calcimimetic-treated chow (0.05% w/w Compound A (3-(2-chlorophenyl)-N-((1R)-1-(3-methyloxy)phenyl)ethyl)-1-propanamine) blended into grain based rat chow with normal calcium and phosphorus content). Average food intake was 50 g/kg/day. At the time of sacrifice, rats were anesthetized with sodium pentobarbitol (100 g/kg, i.p.) and weighed. Blood was obtained for determination of serum creatinine, BUN, intact PTH, Ca and PO₄. The left kidney was removed, weighed and frozen in liquid nitrogen for subsequent batch analyses. The right kidney was perfusion fixed with 4% paraformaldehyde in phosphate buffer (ph 7.4). The tissue was embedded in paraffin for histological studies. Cyst volume density was determined by point count stereology from randomly selected fields from hematoxylin and cosin (H&E) stained slides. Fibrosis was graded semi-quantitatively (scored 0-4+) from picrosirius red stained slides. The reviewer was blinded to the treatment group. Both measure were also standardized by body weight (for cyst volume) or kidney weight (for fibrosis).

Histomorphometric Analysis

Four micron transverse tissue section of the kidney, including cortex and medulla, was stained with hematoxylin-cosin. Five fields/sections, from cortex through outer medulla, were evaluated for the degree of cystic change. Other sections were stained with picrosirius red for the evaluation of fibrosis. Fibrosis was scored (0-4+) for the relative amount of collagen within the interstitium from picrosirius red staining of collagen fibers. These assessments are the standard methods used to assess response to therapy in animal models of kidney disease (Gattone et al, Nature Medicine 9: 1323-6, 2003).

Biochemical Determinations

Plasma urea and creatinine were determined using colorimetric assays (Urea-Sigma 640 creatinine kit). Intact PTH was determined by ELISA; Alpacoa, and Ca and PO₄ were done with using colorimeteric analyses (Sigma).

Study Design

This study was designed to evaluate the effectiveness of the calcimimetics for treatment and prevention of PKD in male Cy/+ rat model. The male was utilized due to the earlier onset of disease not because of gender specificity in efficacy.

Cy−/+ (cystic) male rats were assigned to one of four treatment groups (Table 5) and were phenotyped (by BUN) at 10 weeks and again at 20 weeks to ensure animals were cystic. At 20 weeks, the animals began therapy with the treatment groups listed in the table below. This study was designed to evaluate the efficacy of Compound A (3-(2-chlorophenyl)-N-((1R)-1-(3-(methloxy)phenyl)ethyl)-1-propanamine), a calcimimetic, in male Cy/+ rats after they were already azotemic. Comparator groups included Compound A with calcium in the drinking water versus calcium alone. These comparator groups allow differentiation of the effects of changes in PTH and calcium from a direct action of the calcimimetic.

TABLE 4

| Group | Compound A in diet | 3% Ca Gluconate in water |
|---|---|---|
| 1-control (cystic rats) | No | No |
| 2-compound A only | Yes | No |
| 3-compound A and | Yes | Yes |
| calcium | | |
| 4-calcium only | No | Yes |

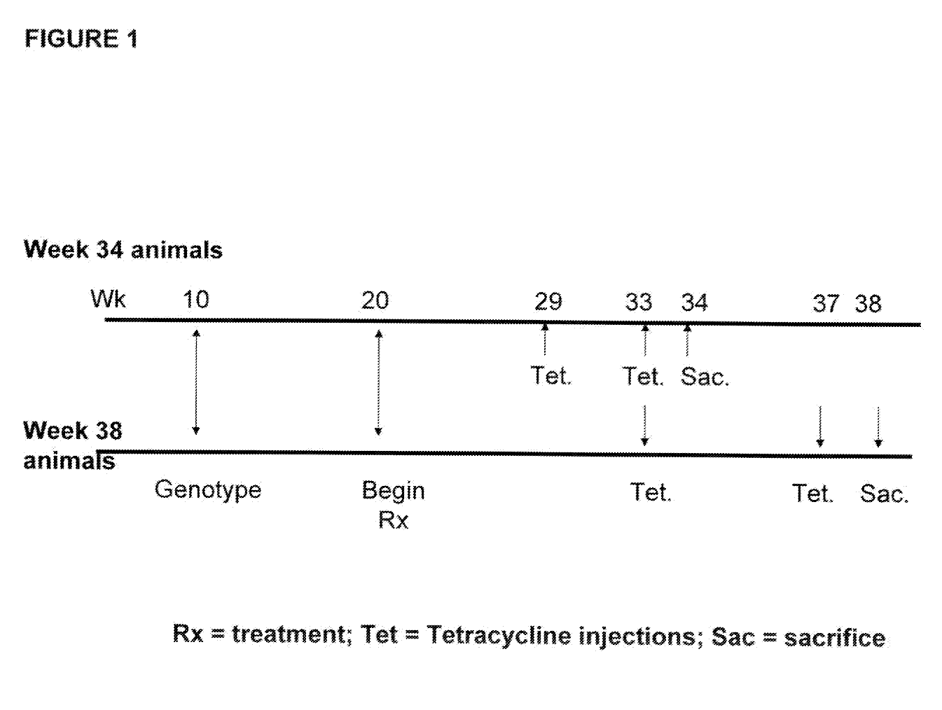

FIG. 1 schematically represents the study design. At 10 weeks, animals were phenotyped. At 20 weeks, animals were started on the treatment arm. Half the animals were sacrificed at 34 weeks, half the animals were sacrificed 38 weeks. At each time point, samples from 8 animals were perfusion fixed with paraformaldehyde for CT scan, bone histology and aorta histology, samples from 8 animals were saline perfused for HCl extraction to determine calcium and phosphate content, and blood samples from 16 animals were collected for blood tests (PTH, calcium, phosphorus, BUN). Animals in the paraformaldehyde fixation group received tetracycline administration four and one week prior to sacrifice to label the bone for morphometric analyses. For the purpose of this application, only the laboratory and kidney end points will be presented.

Statistical Methods and Power Calculations

All data was analyzed by a statistician. The BUN was analyzed only at week 20 and end point (week 34 or 38) and was analyzed by ANCOVA adjusting for baseline (week 20) values. The PTH, calcium, and phosphorus were analyzed at baseline (week 20), a mid point, and end point (week 34, 38). These values were compared by a mixed model adjusting for baseline values and time point. The kidney assessments were only measured at end point at the time of sacrifice, and therefore the measures were compared by ANCOVA and within group comparisons.

### EXAMPLE 2

This Example demonstrates that the calcimimetic Compound A reduces cyst volume and fibrosis.

Table 5 below details the differences in laboratory values at 38 week time point. (±5 mg)

TABLE 5

| | | Compound | Compound | |
|---|---|---|---|---|
| Labs | CKD-Ctl | A | A + Ca | Ca alone |
| Number animals per group | 15 | 16 | 15 | 15 |
| PTH (pg/ml) | 596 ± 685 | 131 ± 156 | 36 ± 21 | 39 ± 23 |
| Calcium (mg/dl) | 9.3 ± 1.0 | 8.2 ± 1.5 | 9.8 ± 1.3 | 11.2 ± 1.5 |
| Phosphorus (mg/dl) | 6.7 ± 1.9 | 5.9 ± 1.4 | 4.6 ± 0.8 | 4.3 ± 0.8 |
| BUN (mg/dl) | 89 ± 22 | 79 ± 21 | 68 ± 8.5 | 69 ± 18 |
| Change in BUN (week 20 to week | 45 ± 20 | 31 ± 15 | 21 ± 8 | 27 ± 11 |
| 38) = Delta BUN (mg/dl) | | | | |

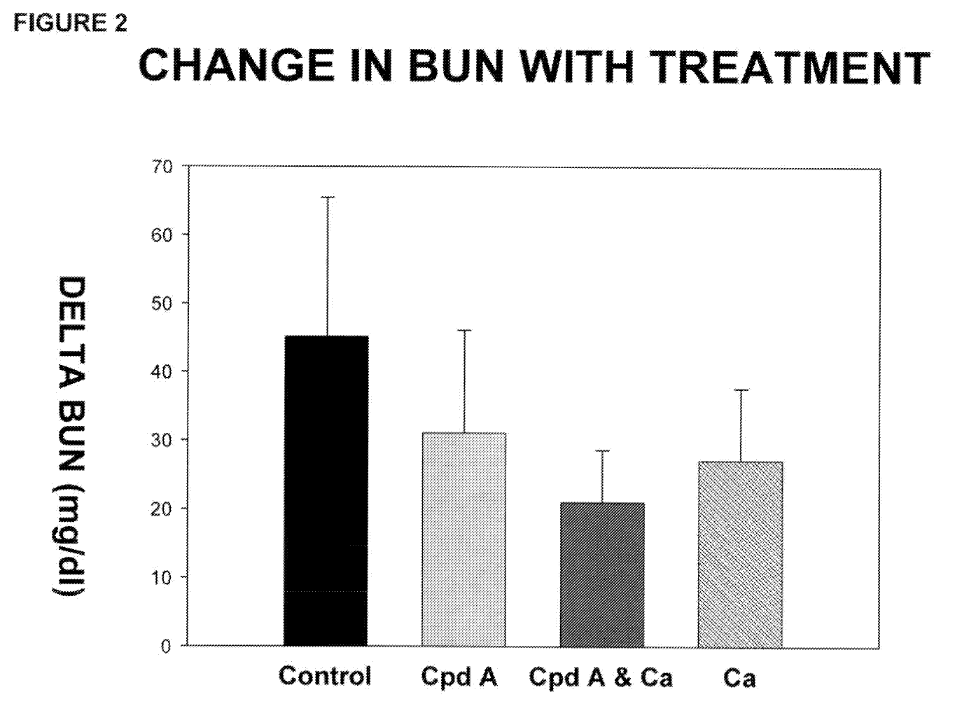

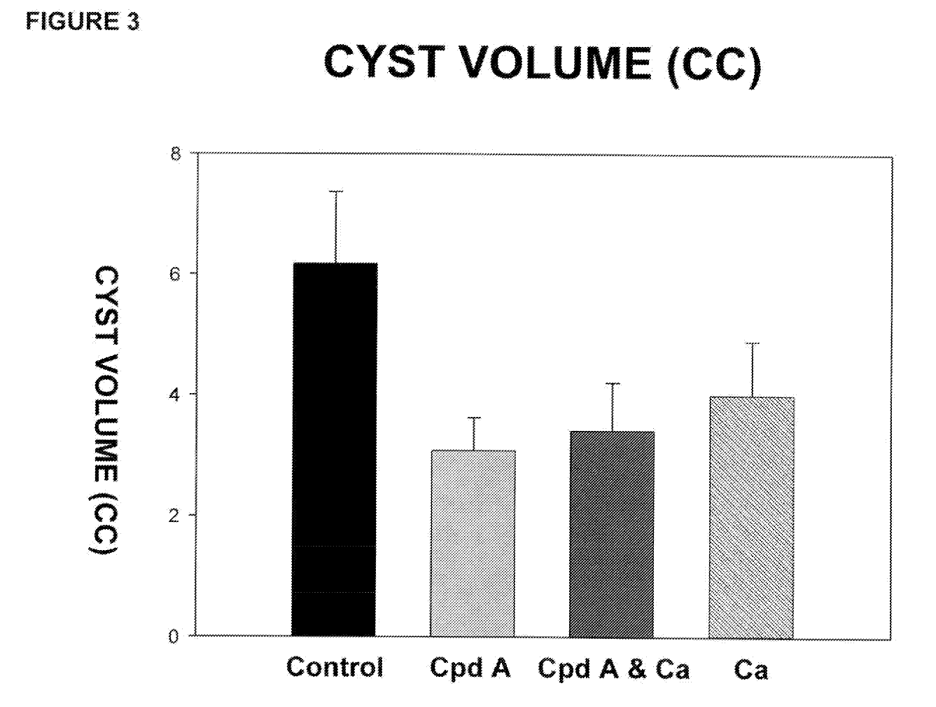

There were differences between the groups at baseline, and a change over the treatment period from 20 to 34 or 20 to 38 weeks. Therefore, by mixed model, after adjustment for baseline value and time, all of the parameters above were significantly different (p<0.001). Within group comparisons revealed differences between all of the groups for PTH and phosphorus. Calcium was different from the control. The BUN was significantly lower in all three treatment groups compared to control after adjustment for baseline values which were slightly higher in the Compound A group. The change in BUN was also significantly lower in all treatment groups compared to control (FIG. 2). Kidney Assessments: (FIGS. 3-6 show the control (no treatment) in black bars, the compound A and compound A+calcium in light and dark gray bars respectively, and the calcium alone in hatched bar. The results are mean±standard deviation.)

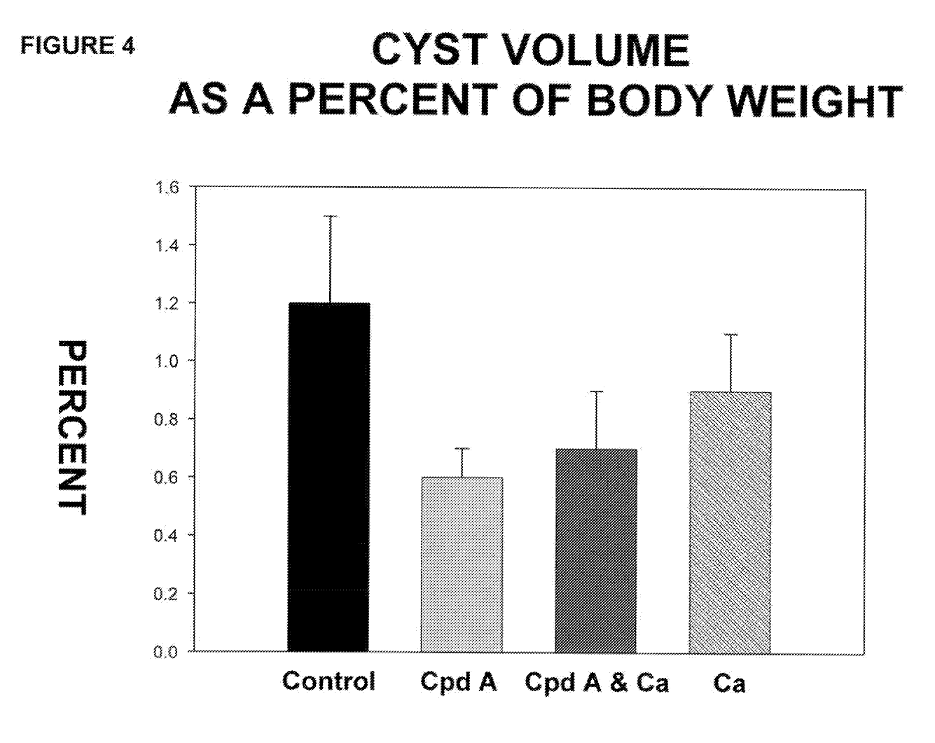

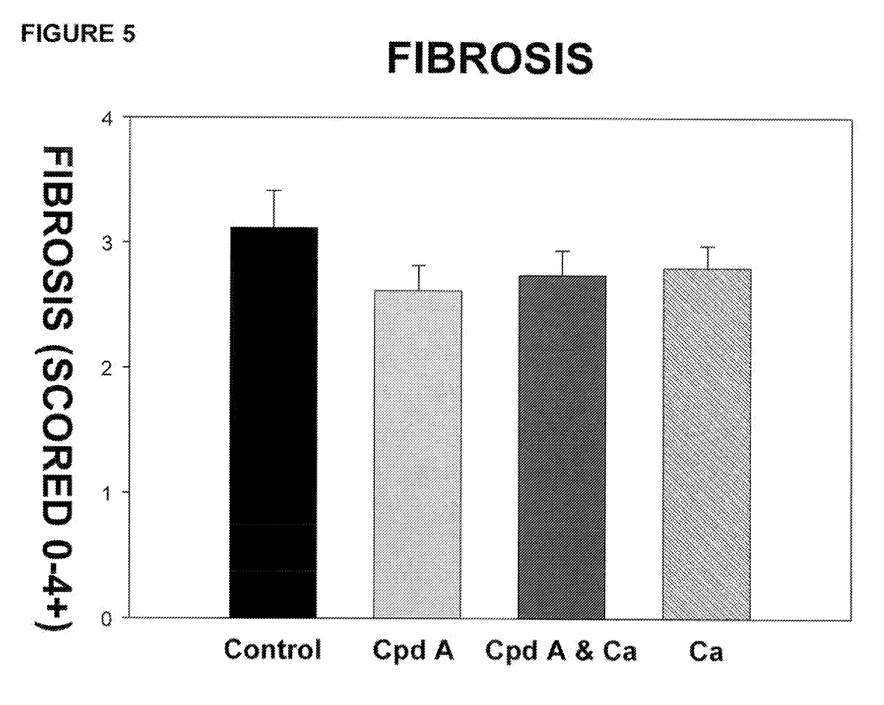

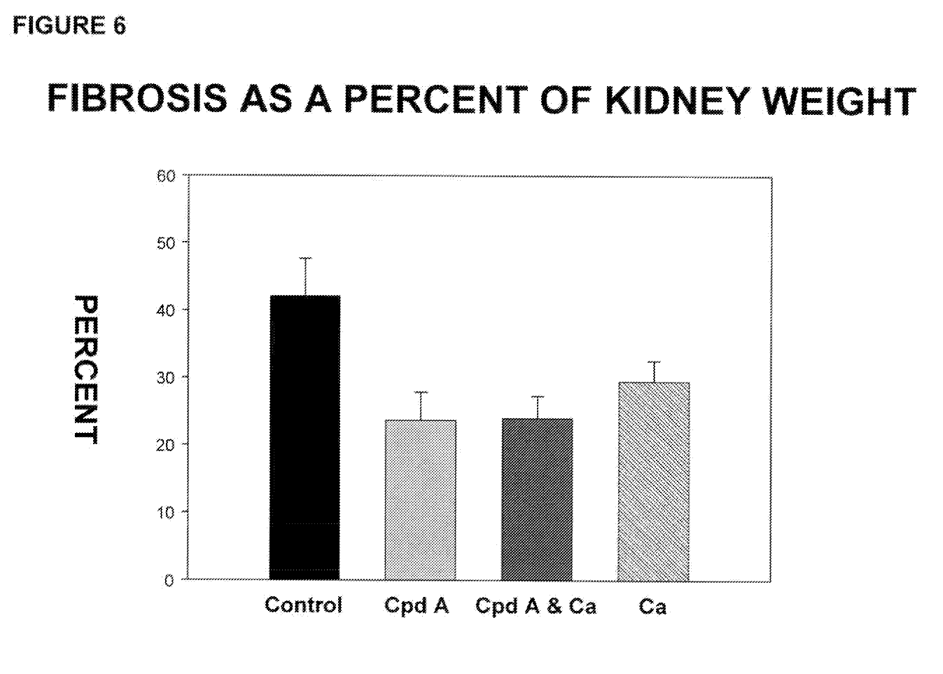

Standard assessments of cyst progression include cyst volume density (Cy*Vv alone as FIG. 3, and as a percent of body weight FIG. 4 below) and the magnitude of fibrosis which progresses with progressive kidney disease (alone as FIG. 5, and weighted for kidney weight in FIG. 6). The latter is a graded semi-quantitative score. As is shown in FIGS. 3-6, all treatments favorably affected (reduced) cyst volume and fibrosis. However, the magnitude was significantly greater in the animals treated with compound A by ANOVA with post group comparisons, all p<0.01.

The actual data is presented in Table 6 below:

TABLE 6

| Kidney Assessment (n = 7-8) | CKD-ctl | Cpd A | Cpd A + Ca | Ca alone |
|---|---|---|---|---|
| Cyst Vol (cc) | 6.2 ± 1.2 | 3.1 ± 0.55 | 3.4 ± 0.8 | 4.0 ± 0.9 |
| Cyst Vol as a % of BW | 1.2 ± 0.3 | 0.6 ± 0.1 | 0.7 ± 0.2 | 0.9 ± 0.2 |
| Fibrosis Score | 3.1 ± 1.0 | 2.6 ± 0.2 | 2.7 ± 0.2 | 2.8 ± 0.2 |
| Total Kidney weight (g) | 10.8 ± 3.0 | 9.5 ± 1.4 | 8.4 ± 1.1 | 10.1 ± 1.4 |

BW—body weight; KW = kidney weight mean ± SD

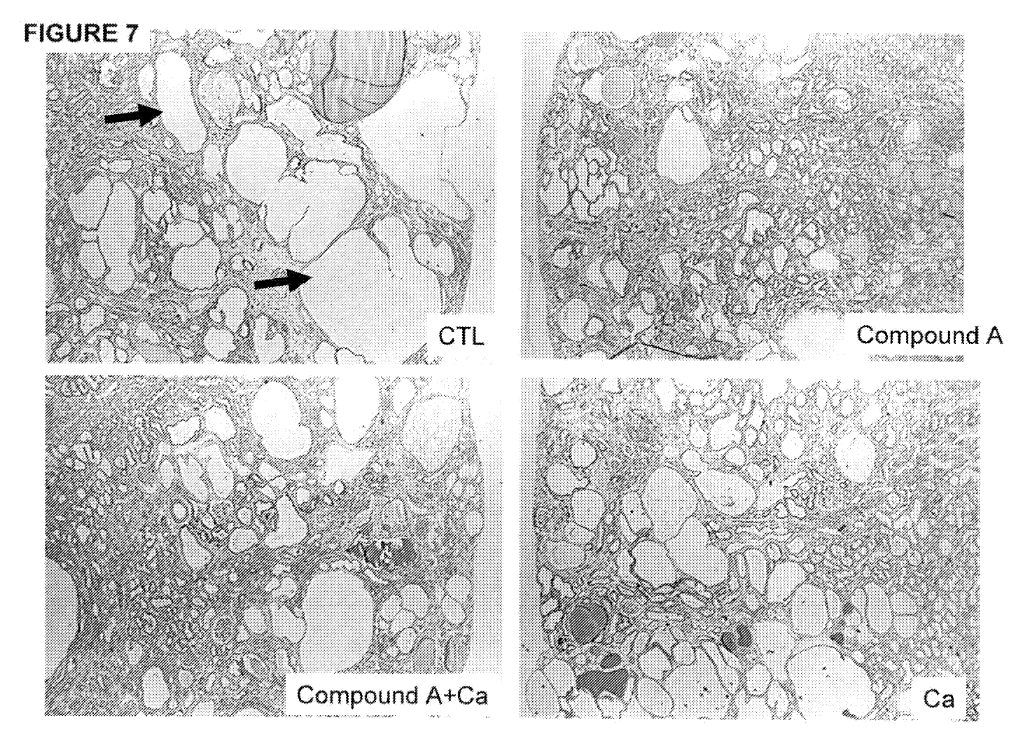

The histologic appearance of the cysts also differ in the treatment groups as shown below in representative pictures from the different treatment groups (FIG. 7): The upper left panel is control cystic animals and there are multiple large cysts (arrows). The upper right panel is from a kidney of an animal treated with Compound A demonstrating a noticeable decrease in cyst size and number. The lower left panel is from animals treated with Compound A+Calcium and the cysts are also smaller in size. The lower right panel is from calcium treated and the cysts are a little smaller than control animals but there appears to be a similar number. The cyst volume density reflects both the number and the overall size of the cysts by determining the volume (in cc) of the kidney of the cysts (CysVv×KW).

These data demonstrate that both cyst volume and fibrosis are significantly reduced in all treatment groups compared to control, however the magnitude of the change is greatest with the Compound A treated groups. Importantly, these changes occurred in the calcimimetic group alone despite a less dramatic reduction in PTH than the calcium treated groups. Therefore, the effects of the calcimimetic Compound A are distinct (and possibly additive) to the effects of lowering PTH. These novel finding support that calcimimetics may be an effective therapy for slowing (and perhaps preventing) the progression of cystic kidney disease in humans.

All publications, patents and patent applications cited in this specification are herein incorporated by reference as if each individual publication or patent application were specifically and individually indicated to be incorporated by reference. All explanations provided herein were provided by way of illustration and not limitation. The various aspects of the invention disclosed and inferred herein should not be limited in any way by theories or hypothesis provided herein to help describe and enable the invention disclosed herein. Although the foregoing invention has been described in some detail by way of illustration and example for purposed of clarity of understanding, it will be readily apparent to those of ordinary skill in the art in light of the teachings of this invention that certain changes and modifications may be made thereto without departing from the spirit or scope of the appended claims.

What is claimed is:

1. A method for treating a cystic kidney disease, comprising administering to a subject in need thereof a therapeutically effective amount of a calcimimetic compound or a pharmaceutically acceptable salt thereof.

2. The method of claim 1, wherein the cystic kidney disease is autosomal dominant polycystic kidney disease (ADPKD).

3. The method of claim 1, wherein the cystic kidney disease is autosomal recessive polycystic kidney disease (ARPKD).

4. The method of claim 1, wherein the cystic kidney disease is acquired renal cystic disease (ARCD).

5. The method of claim 1, wherein the cystic kidney disease is medullary cystic kidney disease (MCKD).

6. The method of claim 1, wherein the cystic kidney disease is nephronophthisis (NPH).

7. The method of claim 1, wherein the cystic kidney disease is multicystic dysplasia, congenital cystic disease, Meckel syndrome, oro-facial-digital syndrome, tuberous sclerosis, Von Hippel-Landau syndrome, cerebro-renal-digital syndrome, genitopatellar syndrome or Bardt-Biedl syndrome.

8. The method of claim 1, further comprising administering a pain medication.

9. The method of claim 8, wherein the pain medication is acetaminophen, NSAID, tramadol, clonidine, a narcotic, or an opioid.

10. The method of claim 1, further comprising administering a medication to reduce blood pressure.

11. The method of claim 10, wherein the medication to reduce blood pressure is an antihypertensive medication.

12. The method of claim 10, wherein the medication to reduce blood pressure is a diuretic.

13. The method of claim 1, further comprising administering an antibiotic.

14. The method of claim 1, further comprising administering EGFR tyrosine, kinase inhibitor, vasopressin V₂ receptor antagonist, MTOR inhibitors, somatostatin agonists, or cdk inhibitors.

15. The method of claim 1, further comprising a surgical treatment.

16. The method of claim 1, further comprising a lifestyle or dietary modification.

17. The method of claim 1, wherein the calcimimetic compound is a compound of Formula I 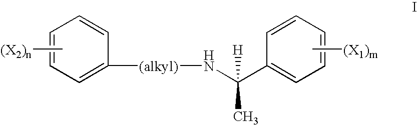
wherein:
X₁ and X₂, which may be identical or different, are each a radical chosen from CH₃, CH₃O, CH₃CH₂O, Br, Cl, F, CF₃, CHF₂, CH₂F, CF₃O, CH₃S, OH, CH₂OH, CONH₂, CN, NO₂, CH₃CH₂, propyl, isopropyl, butyl, isobutyl, t-butyl, acetoxy, and acetyl radicals, or two of X₁ may together form an entity chosen from fused cycloaliphatic rings, fused aromatic rings, and a methylene dioxy radical, or two of X₂ may together form an entity chosen from fused cycloaliphatic rings, fused aromatic rings, and a methylene dioxy radical; provided that X₂ is not a 3-t-butyl radical;
n ranges from 0 to 5;
m ranges from 1 to 5; and
the alkyl radical is chosen from C₁-C₃ alkyl radicals, which are optionally substituted with at least one group chosen from saturated and unsaturated, linear, branched, and cyclic C₁-C₉ alkyl groups, dihydroindolyl and thiodihydroindolyl groups, and 2-, 3-, and 4-piperidinyl groups;
or a pharmaceutically acceptable salt thereof.

18. The method of claim 17, wherein the calcimimetic compound is N-(3-[2-chlorophenyl]-propyl)-R-α-methyl-3-methoxybenzylamine or a pharmaceutically acceptable salt thereof.

19. The method of claim 1, wherein the calcimimetic compounds is cinacalcet or a pharmaceutically acceptable salt thereof.

20. The method of claim 1, wherein the calcimimetic compound is a compound of the Formula II 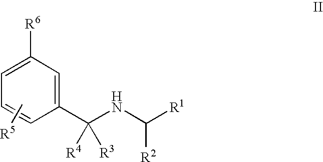
wherein:
R¹ is aryl, substituted aryl, heterocyclyl, substituted heterocyclyl, cycloalkyl, or substituted cycloalkyl;
R² is alkyl or haloalkyl;
R³ is H, alkyl, or haloalkyl;
R⁴ is H, alkyl, or haloalkyl;
each R⁵ present is independently selected form the group consisting of alkyl, substituted alkyl, alkoxy, substituted alkoxy, halogen, —C(═O)OH, —CN, —NR^{d}S(═O)ₘR^{d}, —NR^{d}C(═O)NR^{d}R^{d}, —NR^{d}S(═O)ₘNR^{d}R^{d}, or —NR^{d}C(═O)R^{d};
R⁶ is aryl, substituted aryl, heterocyclyl, substituted heterocyclyl, cycloalkyl, or substituted cycloalkyl;
each R^{a} is, independently, H, alkyl, or haloalkyl;
each R^{b} is, independently, aryl, aralkyl, heterocyclyl, or heterocyclylalkyl, each of which may be unsubstituted or substituted by up to 3 substituents selected from the group consisting of alkyl, halogen, haloalkyl, alkoxy, cyano, and nitro;
each R^{c} is, independently, alkyl, haloalkyl, phenyl or benzyl, each of which may be substituted or unsubstituted;
each R^{d} is, independently, H, alkyl, aryl, aralkyl, heterocyclyl, or heterocyclylalkyl wherein the alkyl, aryl, aralkyl, heterocyclyl, and heterocyclylalkyl are substituted by 0, 1, 2, 3 or 4 substituents selected from alkyl, halogen, alkoxy, cyano, nitro, R^{b}, —C(═O)R^{c}, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}S(═O)ₙR^{c} and —S(═O)ₙNR^{a}R^{a};
m is 1 or 2;
n is 0, 1, or 2; and
p is 0, 1, 2, 3, or 4;
provided that if R² is methyl, p is 0, and R⁶ is unsubstituted phenyl, then R¹ is not 2,4-dihalophenyl, 2,4-dimethylphenyl, 2,4-diethylphenyl, 2,4,6-trihalophenyl, or 2,3,4-trihalophenyl; or a pharmaceutically acceptable salt thereof.

21. The method of claim 20, wherein the calcimimetic compound is N-((6-(methoxyloxy)-4′-(trifluoromethyl)-1,1′-biphenyl-3-yl)methyl)-1-phenylethanamine, or a pharmaceutically acceptable salt form thereof.

22. The method of claim 20, wherein the calcimimetic compounds is (1R)-N-((6-chloro-3′-fluoro-3-biphenylyl)methyl)-1-(3-chlorophenyl)ethanamine, or a pharmaceutically acceptable salt form thereof.

23. The method of claim 20, wherein the calcimimetic compounds is (1R)-1-(6-(methyloxy)-4′-(trifluoromethyl)-3-biphenylyl)-N-((1R)-1-phenylethyl)ethanamine, or a pharmaceutically acceptable salt form thereof.

24. The method of claim 1, wherein the calcimimetic compound is a compound of the Formula III 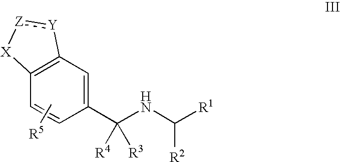
and pharmaceutically acceptable salts thereof, wherein: [symbol]
represents a double or single bond;
R¹ is R^{b};
R² is C₁₋₈alkyl or C₁₋₄haloalkyl;
R³ is H, C₁₋₄haloalkyl or C₁₋₈alkyl;
R⁴ is H, C₁₋₄haloalkyl or C₁₋₄alkyl;
R⁵ is, independently, in each instance H, C₁₋₈alkyl, C₁₋₄haloalkyl, halogen, —OC₁₋₆alkyl, —NR^{a}R^{d} or NR^{d}C(═O)R^{d};
X is —CR^{d}═N—, —N═CR^{d}—, O, S or —NR^{d}—;
when [symbol] is a double bond then Y is ═CR⁶— or ═N—and Z is —CR⁷═ or —N═; and when [symbol] is a single bond then Y is —CR^{a}R⁶— or —NR^{d}— and Z is —CR^{a}R⁷— or —NR^{d}—; and
R⁶ is R^{d}, C₁₋₄haloalkyl, —C(═O)R^{c}, —OC₁₋₆alkyl, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, cyano, nitro, —NR^{a}S(═O)ₘR^{c} or —S(═O)ₘNR^{a}R^{a};
R⁷ is R^{d}, C¹⁻⁴haloalkyl, —C(═O)R^{c}, —OC₁₋₆alkyl, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, cyano, nitro, —NR^{a}S(═O)ₘR^{c} or —S(═O)ₘNR^{a}R^{a}; or R⁶ and R⁷ together form a 3- to 6-atom saturated or unsaturated bridge containing 0, 1, 2 or 3 N atoms and 0, 1 or 2 atoms selected from S and O, wherein the bridge is substituted by 0, 1 or 2 substituents selected from R⁵; wherein when R⁶ and R⁷ form a benzo bridge, then the benzo bridge may be additionally substituted by a 3- or 4-atoms bridge containing 1 or 2 atoms selected from N and O, wherein the bridge is substituted by 0 or 1 substituents selected from C₁₋₄alkyl;
R^{a} is, independently, at each instance, H, C₁₋₄haloalkyl or C₁₋₆alkyl;
R^{b} is, independently, at each instance, phenyl, benzyl, naphthyl or a saturated or unsaturated 5- or 6-membered ring heterocycle containing 1, 2 or 3 atoms selected from N, O and S, with no more than 2 of the atoms selected from O and S, wherein the phenyl, benzyl or heterocycle are substituted by 0, 1, 2 or 3 substituents selected from C₁₋₆alkyl, halogen, C₁₋₄haloalkyl, —OC₁₋₆alkyl, cyano and nitro;
R^{c} is, independently, at each instance, C₁₋₆alkyl, C₁₋₄haloalkyl, phenyl or benzyl;
R^{d} is, independently, at each instance, H, C₁₋₆alkyl, phenyl, benzyl or a saturated or unsaturated 5- or 6-membered ring heterocycle containing 1, 2 or 3 atoms selected from N, O and S, with no more than 2 of the atoms selected from O and S, wherein the C₁₋₆ alkyl, phenyl, benzyl, naphthyl and heterocycle are substituted by 0, 1, 2, 3 or 4 substituents selected from C₁₋₆alkyl, halogen, C₁₋₄haloalkyl, —OC₁₋₆alkyl, cyano and nitro, R^{b}, —OC(═O)R^{c}, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}C(═O)R^{c}, —NR^{a}S(═O)ₘR^{c} and —S(═O)ₘNR^{a}R^{a}; and
m is 1 or 2,
or a pharmaceutically acceptable salt thereof.

25. The method of claim 1, wherein the calcimimetic compound is a compound of Formula IV 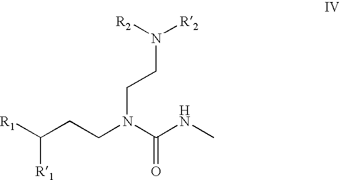
R₁ and R′₁, which may be the same or different, represent an aryl radical, a heteroaryl radical, and aryl or heteroaryl radical substituted by one or more halogen atoms, by one or more hydroxy groups, by one or more linear or branched alkyl or alkoxy radicals containing from 1 to 5 carbon atoms, by one or more trifluoromethyl, trifluoromethoxy, —CN, —NO₂, acetyl carboxyl, carboalkoxy or thioalkyl groups and the oxidised sulfoxide or sulfone forms thereof, trifluoroalkoxy groups,
or R₁ and R′₁ form, with the carbon atom to which they are linked, a cycle of Formula: 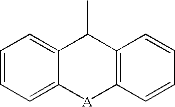
in which A represents a single bond, a —CH₂— group, an oxygen, nitrogen, or sulfur atom,
R₂ and R′₂ form, with the nitrogen atom to which they are linked, a saturated heterocycle containing 4 or 5 carbon atoms, said heterocycle optionally containing a further heteroatom, itself being optionally substituted by a radical R₅ in which R₅ represents a hydrogen atom, a linear or branched alkyl radical containing from 1 to 5 carbon atoms, optionally substituted by an alkoxy or acyloxy radical,
or R₂ and R′₂, which may be the same or different, represent a hydrogen atom, a linear or branched alkyl radical containing from 1 to 5 carbon atoms optionally substituted by a hydroxy or alkoxy radical containing from 1 to 5 carbon atoms,
R₃ represents a thiozolyl, oxazolyl, benzothiazolyl or benzoxazolyl group of Formula: 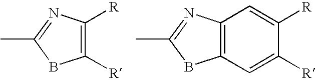
in which B represents an oxygen atom or a sulfur atom, in which R and R′, which may be the same or different, represent a hydrogen atom, a halogen atom, a hydroxy radical, a trifluormethyl radical, a trifluoromethoxy radical, alkyl, alkoxy, alkoxycarbonyl or alkylthio radicals and the oxidised sulfoxide and sulfone form thereof linear or branched containing from 1 to 5 carbon atoms, an aryl or heteroaryl radical, an aryl or heteroaryl radical substituted by one or more groups selected from a halogen atom, a linear or branched alkyl radical containing from 1 to 5 carbon atoms, a trifluoromethyl radical, a trifluoromethoxy radical, a —CN group, an amino, dialklylamino and —NH—CO-alkyl group, an alkylthio group and the oxidised sulfoxide and sulfone form thereof, an alkylsulfonamide —NH—SO₂-alkyl group or by a morpholino group,
or R and R′ on the thiazolyl or oxazolyl group can form a saturated or unsaturated cycle comprising or not comprising one or more optionally substituted heteroatoms,
or a pharmaceutically acceptable salt thereof.

26. The method of claim 25, wherein the calcimimetic compound is 3-(1,3-benzothiazol-2-yl)-1-(3,3-diphenylpropyl)-1-(2-(4-morpholinyl)ethyl)urea or its pharmaceutically acceptable salt thereof.

27. The method of claim 25, wherein the calcimimetic compound is N-(4-(2-((((3,3-diphenylpropyl)(2-(4-morpholinyl)ethyl)amino)carbonyl)amino)-1,3-thiazol-4-yl)phenyl)methanesulfonamide or pharmaceutically acceptable salt thereof.

28. The method of claim 1, wherein the calcimimetic compound is a compound of Formula V 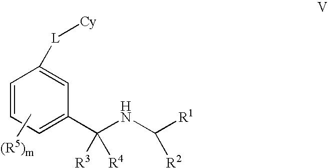
wherein:
R¹ is phenyl, benzyl, naphthyl or a saturated or unsaturated 5- or 6-membered heterocyclic ring containing 1, 2 or 3 atoms selected from N, O and S, with no more than 2 of the atoms selected from O and S, wherein the phenyl, benzyl, naphthyl or heterocyclic ring are substituted by 0, 1, 2 or 3 substituents selected from C₁₋₆alkyl, halogen, C₁₋₄haloalkyl, —C₁₋₆alkyl, cyano and nitro;
R² is C₁₋₈alkyl or C₁₋₄haloalkyl;
R³ is H, C₁₋₄haloalkyl or C₁₋₈alkyl;
R⁴ is H, C₁₋₄haloalkyl or C₁₋₈alkyl;
R⁵ is, independently, in each instance, H, C₁₋₈alkyl, C₁₋₄haloalkyl, halogen, —OC₁₋₆alkyl, —NR^{a}R^{d}, NR^{a}R^{d}, NR^{a}C(═O)R^{d}, substituted or unsubstituted pyrrolidinyl, substituted or unsubstituted azetidinyl, or substituted or unsubstituted piperidyl, wherein the substituents can be selected from halogen, —OR^{b}, —NR^{a}R^{d}, —C(═O)OR^{c}, —C(═O)NR^{a}R^{d}, —OC(═O)R^{c}, —NR^{a}C(═O)R^{c}, cyano, nitro, —NR^{a}S(═O)ₙR^{c} or —S(═O)ₙNR^{a}R^{d};
L is —O—, —OC₁₋₆alkyl-, —C₁₋₆alkylO—, —N(R^{a})(R^{d})—, —NR^{a}C(═O)—, —C(═O)—, —C(═O)NR^{d}C₁₋₆alkyl-, —C₁₋₆alkyl-C(═O)NR^{d}—, —NR^{d}C(═O)NR^{d}—, —NR^{d}C(═O)NR^{d}C₁₋₆alkyl-, —NR^{a}C(═O)R^{c}—, —NR^{a}C(═O)OR^{c}—, —OC₁₋₆alkyl-C(═O)O—, —NR^{d}C₁₋₆alkyl-, —C₁₋₆alkylNR^{d}—, —S—, —S(═O)ₙ—, —NR^{a}S(═O)ₙ, or —S(═O)ₙN(R^{a})—;
Cy is a partially or fully saturated or unsaturated 5-8 membered monocyclic, 6-12 membered bicyclic, or 7-14 membered tricyclic ring system, the ring system formed of carbon atoms optionally including 1-3 heteroatoms if monocyclic, 1-6 heteroatoms if bicyclic, or 1-9 heteroatoms if tricyclic, and wherein each ring of the ring system is optionally substituted independently with one or more substituents of R⁶, C₁₋₈alkyl, C₁₋₄haloalkyl, halogen, cyano, nitro, —OC₁₋₆alkyl, —NR^{a}R^{d}, NR^{d}C(═O)R^{d}, —C(═O)OR^{c}, —C(═O)NR^{a}R^{d}, —OC(═O)R^{c}, —NR^{a}C(═O)R^{c}, —NR^{a}S(═O)ₘR^{c} or —S(═O)ₘNR^{a}R^{d};
R⁶ is a partially or fully saturated or unsaturated 5-8 membered monocyclic, 6-12 membered bicyclic, or 7-14 membered tricyclic ring system, the ring system formed of carbon atoms optionally including 1-3 heteroatoms if monocyclic, 1-6 heteroatoms if bicyclic, or 1-9 heteroatoms if tricyclic, and wherein each ring of the ring system is optionally substituted independently with one or more substituents of C₁₋₈alkyl, C₁₋₄haloalkyl, halogen, cyano, nitro, —OC₁₋₆alkyl, —NR^{a}R^{d}, NR^{d}(═O)R^{d}, —(═O)OR^{c}, —C(═O)NR^{a}R^{d}, —OC(═O)R^{c}, —NR^{a}C(═O)R^{c}, —NR^{a}S(═O)ₘR^{c} or —S(═O)ₘNR^{a}R^{d};
R^{a} is, independently, at each instance, H, C₁₋₄haloalkyl, C₁₋₆alkyl, C₁₋₄alkenyl, C₁₋₆alkylaryl or arylC₁₋₆alkyl;
R^{b} is, independently, at each instance, C₁₋₈alkyl, C₁₋₄haloalkyl, phenyl, benzyl, naphthyl or a saturated or unsaturated 5- or 6-membered heterocyclic ring containing 1, 2 or 3 atoms selected from N, O and S, with no more than 2 of the atoms selected from O and S, wherein the phenyl, benzyl, naphthyl or heterocyclic ring are substituted by 0, 1, 2 or 3 substituents selected from C₁₋₆alkyl, halogen, C₁₋₄haloalkyl, —OC₁₋₆alkyl, cyano and nitro;
R^{c} is, independently, at each instance, C₁₋₆alkyl, C₁₋₄haloalkyl, phenyl or benzyl;
R^{d} is, independently, at each instance, H, C₁₋₆alkyl, C₁₋₆alkenyl, phenyl, benzyl, naphthyl or a saturated or unsaturated 5- or 6-membered heterocycle ring containing 1, 2 or 3 atoms selected from N, O and S, with no more than 2 of the atoms selected from O and S, wherein the C₁₋₆alkyl, phenyl, benzyl, naphthyl, and heterocycle are substituted by 0, 1, 2, 3 or 4 substituents selected from C₁₋₆alkyl, halogen, C₁₋₄haloalkyl, —OC₁₋₆alkyl, cyano and nitro, R^{b}, —C(═O)R^{c}, —OR^{b}, —NR^{a}R^{b}, —C(═O)OR^{c}, —C(═O)NR^{a}R^{b}, —OC(═O)R^{c}, —NR^{a}C(═O)R^{c}, —NR^{a}S(═O)ₘR^{c} and —S(═O)ₘNR^{a}R^{a};
m is 1 or 2;
n is 1 or 2;
provided that if L is —O— or —OC₁₋₆alkyl-, then Cy is not phenyl;
or a pharmaceutically acceptable salt thereof.

29. The method of claim 1, wherein the subject is a mammal.

30. The method of claim 1, wherein the subject is human.
